(19) United States
(12) Patent Application Publication
(10) Pub. No.: US 2002/0090388 A1
(43) Pub. Date: Jul. 11, 2002

(54) Intravascular drug delivery device and use therefor

(76) Inventors: H. David Humes, Ann Arbor, MI (US); Evangelos Tziampazis, Plymouth, MI (US)

Correspondence Address: TESTA, HURWITZ & THIBEAULT, LLP, HIGH STREET TOWER, 125 HIGH STREET, BOSTON, MA, 02110, US

(21) Appl. No.: 09/999,210

(22) Filed: Nov. 30, 2001

Related U.S. Application Data
(60) Provisional application No. 60/250,746, filed on Dec. 1, 2000.

(51) Int. Cl. A61K 9/22

(52) U.S. Cl. 424/422; 604/891.1

(57) ABSTRACT

Disclosed is an implantable drug delivery device for delivering a pre-selected drug directly into the systemic circulation of an animal. The device comprises an anchor immobilizable to an inner wall of an intact blood vessel. The device also comprises a drug containing reservoir that is retained in place within the blood vessel by the immobilized anchor. The reservoir may include, for example, a drug containing osmotic pump or a drug permeable capsule having disposed therein drug containing particles, which release the drug directly into blood passing the reservoir. The invention also provides a minimally invasive method for introducing into a blood vessel and, optionally, removing from the blood vessel the drug delivery device of the invention.

### CROSS-REFERENCE TO RELATED APPLICATIONS

The present application claims priority to, and the benefit of U.S. Ser. No. 60/250,746, the entire disclosure of which is incorporated herein by reference.

### FIELD OF THE INVENTION

The present invention relates generally to an implantable, intravascular drug delivery device. More particularly, the invention relates to an implantable, intravascular drug delivery device for sustained delivery of a drug directly into systemic circulation of an animal, and to procedures for implanting and retrieving the device from the vasculature.

### BACKGROUND OF THE INVENTION

The development of sustained drug delivery devices is still ongoing. See, for example, Langer (1998) NATURE 392, Supp. 5-10. For example, drug can be conjugated with polymers which, when implanted, are then degraded, for example, by proteolytic enzymes or by hydrolysis, to gradually release the drug into an animal. Similarly, drug can be trapped throughout insoluble matrices which can then be administered to an animal. Drug is released via diffusion out of and/or erosion of the matrices. Alternatively, drug can be encapsulated within semi-permeable membranes or liposomes which are then administered to the animal. Following administration, the drug is released either by diffusion through the membranes or via breakdown of the membrane or liposome to release its contents. These approaches, however, have generally been used when the device is implanted at an extravascular, not an intravascular location within a recipient.

Most traditional implantable sustained drug delivery devices include one or more insoluble components. This raises several problems if the drug is to be introduced into the systemic circulation. For example, there is a significant risk that insoluble components placed within the vasculature may cause one or more potentially catastrophic embolisms. See, for example, Gibaldi (1991) BIOPHARMACEUTICS AND CLINICAL PHARMACOKINETICS, Lea & Febiger, London, 4^{th} ed.

Consequently, the foregoing sustained drug delivery devices, generally are introduced into extravascular locations, utilizing, for example, intramuscular, subcutaneous, oral and parenteral routes. However, a significant drawback to such implantable sustained drug delivery devices is their limited ability, because of significant problems with mass transfer, to deliver drugs reliably to the bloodstream. One approach to alleviate this limitation is to induce vascularization around the implanted drug delivery device (see, for example, U.S. Pat. Nos. 4,820,626 and 5,433,508).

Moreover, under certain circumstances, for example, in order to achieve targeted tissue delivery or in view of drug instability and/or toxicity, it maybe necessary to deliver the drug directly into the blood stream. To date, direct drug delivery generally has been achieved via indwelling intravenous catheters that deliver a drug from a reservoir located outside the vasculature, for example, at an intracorporeal but extravascular location, or most frequently, at an extracorporeal location. An example of the former system is where a catheter connected to a subcutaneously implanted drug containing osmotic pump delivers the drug into the blood stream. An example of the latter system is where a drug, for example, the prostaglandin prostacyclin, is administered continuously from an external reservoir via an infusion pump (wearable or bed-side) and catheter directly into the vena cava of a patient suffering, for example, from primary pulmonary hypertension. Unfortunately, these systems typically are implanted via invasive medical procedures and suffer serious limitations in terms of risk of infection, operation errors, patient compliance, and compromised patient quality of life.

It is an object of the invention to provide an implantable, intravascular drug delivery device suitable for the long-term intravenous delivery of a large variety of drugs directly into systemic circulation. It is another object of the invention to provide minimally invasive procedures for introducing into the lumen of a blood vessel and/or retrieving from the lumen of a blood vessel one or more components of the drug delivery device.

### SUMMARY OF THE INVENTION

The present invention provides an implantable, intravascular drug delivery device for sustained delivery of at least one pre-selected drug directly into the systemic circulation of an animal. The drug delivery device may be implanted into the vasculature using non invasive or minimally invasive surgical procedures. Once implanted, the drug delivery device safely delivers the pre-selected drug directly into the blood stream of the recipient over a prolonged period of time. Use of the present device and method provides an easy and reproducible system for delivering therapeutically effective amounts of a pre-selected drug directly into the blood stream of an animal. The device preferably is used for drug delivery in mammals, more preferably in primates, and most preferably in humans.

In one aspect, the intravascular drug delivery device comprises an anchor adapted for immobilization to an inner wall of a blood vessel, in particular, an inner wall of an intact blood vessel. The anchor is designed such that when immobilized in situ, the anchor permits blood in the vessel to pass therethrough. The device further comprises a cell-free drug containing reservoir that is retained in place in the blood vessel by the immobilized anchor, and releases the pre-selected drug into blood passing the reservoir at the implantation site. The drug delivery device may be implanted via non-invasive or minimally invasive methods, for example, via a catheter threaded from a peripheral vascular location, and once implanted can deliver the drug or drugs of interest into systemic circulation over prolonged periods of time. Furthermore, once depleted of drug, or whenever desired, for example, to terminate or modify a treatment regime, the reservoir may be removed and, if appropriate, replaced with another drug containing reservoir to restart therapy.

The term “systemic circulation” as used herein is understood to mean any blood vessel within an animal, for example, an artery, vein, arteriole, or venule, that provides a blood supply to a tissue or other locus.

The term “pre-selected drug” as used herein is understood to mean any physiologically or pharmacologically active substance capable of producing a localized or systemic therapeutic effect when administered to an animal, and includes (i) any active drug and (ii) any drug precursor that may be metabolized within the animal to produce an active drug. It is understood that the definition also embraces combinations of drugs, combinations of drug precursors, and combinations of a drug with a drug precursor. The drug may include, for example, a peptide, a protein, a nucleic acid (for example, deoxyribonucleic acid and/or ribonucleic acid), a peptidyl nucleic acid, fatty acid (for example, prostaglandin), an organic molecule and an inorganic molecule, that has therapeutic value, i.e., elicits a desired effect, when administered to an animal. A pre-selected drug can include, for example: a hormone or synthetic hormone, for example, insulin or human growth hormone, an anti-infective agent, for example, an antibiotic, an antiviral, and an anti-malarial; a chemotherapeutic agent, for example, 5-fluorouracil and cisplatin; an autonomic drug, for example, an anticholinergic agent, adrenergic agent, andrenergic blocking agent, and a skeletal muscle relaxant; a blood formation or blood coagulation modulating agent, for example, an anti-anemia drug, coagulant and an anticoagulant, hemorrhagic agent, and a thrombolytic agent; a cardiovascular drug, for example, a cardiac drug, hypotensive agent, vasodilating agent, inotropic agent, β-blocker, and a sclerosing agent; central nervous system agent, for example, an analgesic, an antipyretic, and an anticonvulsant; or immunomodulating agent, for example, etanercept, or an immunosuppressant; an anti-inflammatory agent such as interferon y or a cytokine such as IL-10 and IL-13; an anti-obesity agent such as leptin; an anti-lipemic agent such as an inhibitor of hydroxymethylglutaryl coenzyme A (HMG-CoA) reductase such as atorvastatin; an anti-emetic agent, such as, cisapride and metoclopramide; an anti-migraine medication, such as, imitrex; a chelating agent, such as, the iron chelator desferoxamine; and a contraceptive or fertility agent.

The term “anchor” as used herein is understood to mean any structure immobilizable to an inner wall of a blood vessel, which when immobilized in the blood vessel does not occlude or prevent blood flow through the vessel. The anchor comprises at least one element biased in a radially outward direction when immobilized in the lumen of a blood vessel. In other words, the anchor comprises an element that creates a radial interference fit with the inner wall of the blood vessel.

In one embodiment, the anchor may comprise a stent or stent-like element that can be expanded until it becomes radially biased against the inner wall of the blood vessel. Furthermore, the anchor may comprise a barbed or hooked element which can bind the inner wall of the blood vessel. For example, such an anchor may comprise a head and a plurality of barbed or hooked filaments attached to and extending radially from the head such that the filaments are capable of opening umbrella-like until the barbs or hooks located at the end of each filament engage the inner wall of the blood vessel.

In another embodiment, the anchor is an embolism anti-migration filter, such as a blood clot anti-migration filter. A variety of blood clot anti-migration filters useful in the practice of the invention are known in the art. A currently preferred anchor is an anti-migration filter known as a “Greenfield® vena cava filter”. Useful Greenfield® vena cava filters are described in U.S. Pat. Nos. 4,817,600 and 5,059,205. Typically, Greenfield filters comprise a head attached to a plurality of spring biased filaments which, when inserted into the lumen of a blood vessel open, umbrella-like, to contact and grip the inner wall of the blood vessel.

In another embodiment, the anchor may further comprise a receptacle for receiving the reservoir. Moreover, the receptacle may further comprise a locking mechanism to engage and lock the reservoir to the anchor. It is contemplated that both the anchor and the reservoir may comprise interlocking components that mate with one another to lock the reservoir to the anchor.

The term “cell-free reservoir” as used herein is understood to mean any element, free or substantially free of cells (irrespective of whether any residual cells are viable or dead), that is dimensioned to fit within the lumen of a blood vessel, which, when introduced into the blood vessel, does not occlude or prevent blood flow through the vessel. Furthermore, the reservoir is capable of releasing one or more drugs into blood passing the reservoir in the blood vessel. The reservoir further comprises a wall that at least partially defines an inner volume for retaining the drug and at least one pore to permit release of the drug into the blood system.

In a preferred embodiment, the drug is released gradually from the reservoir at a desired rate and over a period of time suitable to ameliorate the symptoms of a disorder. Drug release may occur over a period of weeks, and more preferably over a period of months. In some cases the drug may be released over a period of years.

In one embodiment, the reservoir is an active drug delivery system, for example, a pump system. Commercially available pump systems, include, for example, an osmotic pump that provides sustained drug release at a predetermined rate over a predetermined period of time, and a micromotor pump designed to provide one or more drug release profiles, that may be pre-programmed prior to implantation or programmed post-implantation with the aid of an extracorporeal controller, as required by the physician.

In another embodiment, the reservoir is a passive drug delivery system. The passive drug delivery system can include, for example, a reservoir that comprises a drug permeable capsule having disposed therein drug-containing particles, for example, microencapsulated or gel-immobilized drug, which are adapted to release the drug. The drug permeable capsule preferably is defined by, for example, a semi-permeable membrane. The semi-permeable membrane can contain one or more pores dimensioned to permit passage of the drug therethrough while at the same time preventing passage of the particles through the pores. Polymers useful in producing biocompatible semi-permeable membranes of the present invention include, but are not limited to, polyvinylchloride, polyvinylidene fluoride, polyurethane isocyanate, alginate, cellulose and cellulose derivatives (for example, cellulose acetate, cellulose diacetate, cellulose triacetate, cellulose nitrate), polysulfone, polyarylate, polycarbonate, polystyrene, polyurethane, polyvinyl alcohol, polyacrylonitrile, polyamide, polyimide, polymethylmethacrylate, polyethylene oxide, polytetafluoroethylene or copolymers thereof.

The drug-containing particles can be engineered to provide desired drug delivery profiles, for example, through a combination of polymer coatings that erode and release the drug at varying rates. Furthermore, in addition to the use of drug delivery devices whereby the drug is preloaded into the reservoir prior to implantation, the invention provides methods and compositions whereby the reservoir can be implanted while empty and then loaded with drug in situ. The latter permits the use of large reservoirs that can be implanted and retrieved via a catheter but yet are able to deliver large volumes and/or amounts of drugs. Furthermore, the reservoir may also be recharged or refilled after the drug has been depleted by loading new drug into the reservoir by means of a catheter connected at one end to the reservoir and the other end connected to an additional new source of drug. The additional new source of drug may be a reservoir, a pump, and/or a vascular access port, for example, disposed subcutaneously in the recipient.

It is contemplated that a variety of device configurations may be useful in the practice of the invention. For example, the reservoir may be retained upstream of the anchor, for example, when the reservoir is of a size such that it cannot pass through the anchor. Alternatively, the reservoir may be located downstream of the anchor but retained in place by an attachment means, for example, via a hook or tether extending from the anchor to the reservoir or via an interlock mechanism. In addition, it is contemplated that the reservoir and anchor may be configured such that a portion of the reservoir may be located upstream of the anchor with another portion located downstream of the anchor. This type of configuration can be facilitated, for example, via an interlock or locking mechanism between the anchor and reservoir, or where the reservoir is wedge-like in shape, such that the narrow end of the wedge passes through the anchor but the larger end contacts the anchor thereby to prevent passage of the entire reservoir through the anchor.

In a preferred embodiment, the reservoir comprises a locking mechanism that mates with a reciprocal locking mechanism on or at the anchor to engage and lock the anchor and reservoir to one another. It is contemplated that a variety of locking mechanisms may be useful in the practice of the invention.

Furthermore, the reservoir may contain more than one drug, for example, two, three, or four separate drugs, for release therefrom. For example, the reservoir may contain a combination of inotropes, such as dopamine and dobutamine, which may be combined to ameliorate the symptoms of congestive heart failure, or antibiotics, such as vancomycin and ceftazidime, which may be used in combination to treat an infection, for example, an infection of the central nervous system.

In another aspect, the invention provides a method for introducing into a blood vessel of an animal, a device for delivering a pre-selected drug directly into systemic circulation. The method comprises the steps of (a) immobilizing an anchor to an inner wall of an intact blood vessel, which when immobilized permits blood in the vessel to pass therethrough and (b) introducing into the blood vessel a cell-free reservoir containing the pre-selected drug, such that when introduced into the blood vessel, the reservoir is retained in position by the anchor and releases the pre-selected drug into blood passing the reservoir. Furthermore, in an additional step, the reservoir is locked to the anchor after the anchor has been immobilized in the blood vessel.

In this method, the anchor, the reservoir, or both the anchor and reservoir, may be introduced into the blood vessel via a catheter. In one such procedure the anchor and/or the reservoir may be introduced via catheter into the mammal via a femoral or jugular vein and then immobilized in a natural vein, for example, an inferior vena cava, a superior vena cava, a portal vein or a renal vein, or alternatively, immobilized in a synthetic vein, for example, a vein developed from a surgically-constructed arteriovenous fistula. It is contemplated that selection of appropriate sites for introduction and immobilization of the device is within the level of skill in the art.

In another aspect, the invention provides an anchor for implantation into an intact blood vessel of an animal. The anchor comprises a first element attached to a second element. The first element is adapted for immobilization to an inner wall of the blood vessel and comprises at least one member biased in a radially outward direction when immobilized in the blood vessel. The second element forms a receptacle for receiving a drug delivery reservoir member of a predetermined geometry and/or configuration. In one embodiment, the first element is located proximal to the second element, and, more preferably, the first element is located at a proximal end of the anchor and the second element is located at a distal end of the anchor.

In one embodiment, the first element is a stent that can be expanded radially outward to contact an inner wall of an intact blood vessel. Alternatively, the first element is a barb that can contact and engage an inner wall of the intact blood vessel.

In another embodiment, the second element may further comprise an interlocking mechanism for mating with and engaging a reciprocal interlocking mechanism of the reservoir to lock the reservoir to the anchor. Preferably, the interlocking mechanism of the second element comprises an annular member having an inner wall that defines a bore running through the annular member, in which the inner wall further defines a groove perpendicular to the bore for engaging a reciprocal interlocking mechanism interlock of the reservoir.

In another embodiment, the first element may be connected to the second element via a third element interposed between the first and second elements. The third element may be a rod or filament attached at one end to the first element and attached at its opposite end to the second element.

In another aspect, the invention provides a drug delivery reservoir for implantation into an intact blood vessel of an animal. The reservoir comprises a first element attached to a second element. The first element forming an interlocking mechanism for engaging a reciprocal interlocking mechanism of an anchor immobilizable to an inner wall of an intact blood vessel. The second element comprises a wall that at least partially defines an inner volume for retaining the drug and defines at least one pore dimensioned to permit the drug to exit the reservoir into the blood stream.

In one embodiment, the interlocking mechanism of the first element comprises an annular member having an outer wall, in which a first portion of the outer wall has a first radial dimension, and a second portion of the outer wall has a radial dimension larger than that of the first portion. In another embodiment, the portion of the outer wall having the second radial dimension mates with and engages a groove disposed within a reciprocal interlocking mechanism on the anchor.

In another embodiment, the second element can comprise either an active drug delivery mechanism, for example, an osmotic pump or a micropump, or a passive drug delivery device, for example, a drug permeable capsule having disposed therein drug containing particles that release drug into the blood stream.

In addition, the invention provides an intravascular drug delivery device for delivering a pre-selected drug into systemic circulation of an animal. The device comprises an extravascular element such as a reservoir, a pump, and/or a vascular access port capable of having pre-selected drug disposed therein and a conduit. The conduit has a first end and a second end. The first end can be in fluid communication with the extravascular element to permit the pre-selected drug to enter the conduit, and the second end of the conduit can be anchorable in the lumen of a blood vessel and can permit the pre-selected drug to flow out of the conduit and into the blood stream. The second end of the conduit, when anchored in the blood vessel, can be located in the center of the lumen of the blood vessel. The second end of the conduit can be attached to a blood permeable element anchorable to an inner wall of a blood vessel. The conduit can also include an integral anchor adjacent to the second end. The integral anchor can include at least one element biased in a radially outward direction, anchorable to an inner wall of a blood vessel, and/or can include a stent, and/or can include an outwardly extending barb.

### BRIEF DESCRIPTION OF THE DRAWINGS

The present invention will now be more particularly described with reference to and as illustrated in, but in no manner limited to, the accompanying drawings, in which:

FIGS. 1A-E are schematic illustrations of exemplary drug delivery devices located within the lumen of a blood vessel, where the direction of blood flow through the vessel is depicted by an arrow;

FIGS. 2A-C are schematic illustrations showing an exemplary anchor (FIG. 2A), an exemplary reservoir (2B), and the exemplary anchor interlocked with an exemplary reservoir (FIG. 2C);

FIGS. 3A-B are schematic illustrations of an exemplary drug delivery device of the invention (FIG. 3A), and an exemplary drug delivery device in relation to a device for introducing and/or removing the reservoir member (FIG. 3B);

FIGS. 4A-C depict a three-dimensional schematic illustration of an exemplary anchor useful in the practice of the invention (FIG. 4A), a side-sectional schematic illustration of the anchor (FIG. 4B), and a top plan illustration of the anchor (FIG. 4C);

FIGS. 5A-C depict a three-dimensional schematic illustration of an exemplary anchor useful in the practice of the invention (FIG. 5A), a side-sectional illustration of such an anchor (FIG. 5B), and a top plan illustration of such an anchor (FIG. 5C);

FIG. 6 is a side-sectional schematic illustration depicting an exemplary reservoir useful in the practice of the invention;

FIGS. 7A-B are cross-sectional views of two exemplary passive drug release reservoirs useful in the practice of the invention;

FIGS. 8A-B are side-sectional schematic illustrations of two exemplary reservoirs for passive drug delivery;

FIGS. 9A-D are side-sectional schematic illustrations showing the steps during which an exemplary reservoir is introduced into a blood vessel and engaged via an exemplary anchor immobilized within a blood vessel; and

FIGS. 10A-C are side-sectional schematic illustrations showing the introduction of an empty reservoir into a blood vessel and its filling with drug in situ.

In the drawings, like characters in the respective drawings indicate corresponding parts.

### DETAILED DESCRIPTION OF THE INVENTION

In its most general application, the present invention provides an implantable, intravascular drug delivery device for sustained delivery of a pre-selected drug into the systemic circulation of an animal. The device of the invention is adapted for direct implantation into a blood vessel, preferably using a catheter. After implantation, the drug delivery device releases the pre-selected drug into the blood stream of the recipient.

The drug delivery device comprises an anchor component and a reservoir component. The anchor is dimensioned for insertion into the lumen of an intact blood vessel. Once introduced to a desired location, the anchor is immobilized to an inner wall of the blood vessel. The anchor is designed such that when immobilized to the wall of the blood vessel, the element permits blood in the vessel to pass therethrough. The reservoir also is dimensioned for insertion into the lumen of the blood vessel. The reservoir is retained in situ via the anchor. The reservoir, although free or substantially free of cells, contains at least one drug that is released gradually into the blood passing the reservoir member. Upon entry into the blood stream, the drug becomes disseminated rapidly throughout the vasculature of the recipient and/or is taken up preferentially by a diseased tissue downstream of the device. Proper operation of the drug delivery device requires, therefore, that it does not occlude the blood vessel, i.e., the device does not prevent passage of blood through the blood vessel.

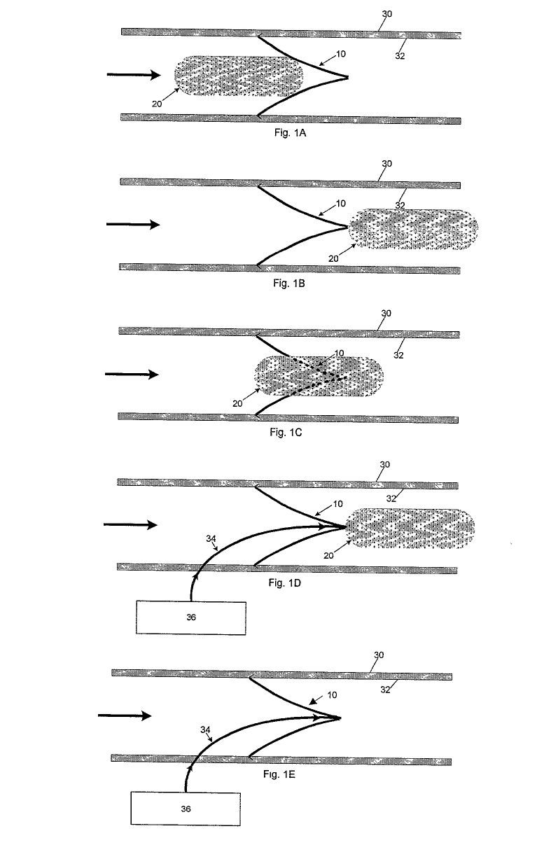

The device of the invention is described in greater detail with reference to the drawings, which are provided for purposes of illustration and are not meant to be limiting in any way. FIG. 1 shows side view illustrations of exemplary configurations of drug delivery devices of the invention. In FIG. 1, the arrows represent the direction of blood flow. FIG. 1A depicts anchor 10 and reservoir 20, where anchor 10 is immobilized in blood vessel 30 via an inner wall 32 of intact blood vessel 30. The reservoir 20 is located upstream of the immobilized anchor 10. In FIG. 1B, reservoir 20 is located downstream of anchor 10 immobilized to an inner wall 32 of an intact blood vessel 30. In FIG. 1C, the reservoir 20 is positioned relative to anchor 10 immobilized to an inner wall 32 of a blood vessel such that a portion of the reservoir 20 is located upstream of anchor 10 and a portion of the reservoir 20 is located downstream of anchor 10.

In FIG. 1D (which is similar to FIG. 1B), the reservoir 20 is located downstream of anchor 10 immobilized to an inner wall 32 of an intact blood vessel 30. The device is configured to permit the loading of drug into reservoir 20 from extravascular element 36 (for example, a reservoir, a pump, and/or a vascular access port) located extravascularly, for example, subcutaneously, via catheter 34 which is connected at one end to extravascular element 36 and at its other end to reservoir 20. Such an extravascular element also can be used in combination with an intravascular reservoir located with respect to the anchor as shown in FIGS. 1A and 1C.

The mechanism by which reservoir 20 is retained by anchor 10 may vary depending upon the relative configuration of the components of the device. For example, in the configurations shown in FIGS. 1A and 1C, the reservoir 20 may be retained in position by contacting anchor 10 where reservoir 20 is dimensioned such that it is too large to pass entirely through the anchor 10. However, it is contemplated that in the configurations shown in FIGS. 1A-1C, reservoir 20 may be locked or otherwise physically tethered to anchor 10 via a locking or tethering mechanism.

In FIG. 1E, anchor 10 is immobilized to an inner wall 32 of intact blood vessel 30. One end of catheter 34 is attached to extravascular element 36 (for example, a reservoir, a pump, and/or a vascular access port). The other end of catheter 34 is attached to anchor 10 which immobilizes catheter 34 within the blood vessel to minimize contact with the inner wall 32 of blood vessel 30. In this device, drug is delivered from extravascular element 36 directly into blood vessel 30.

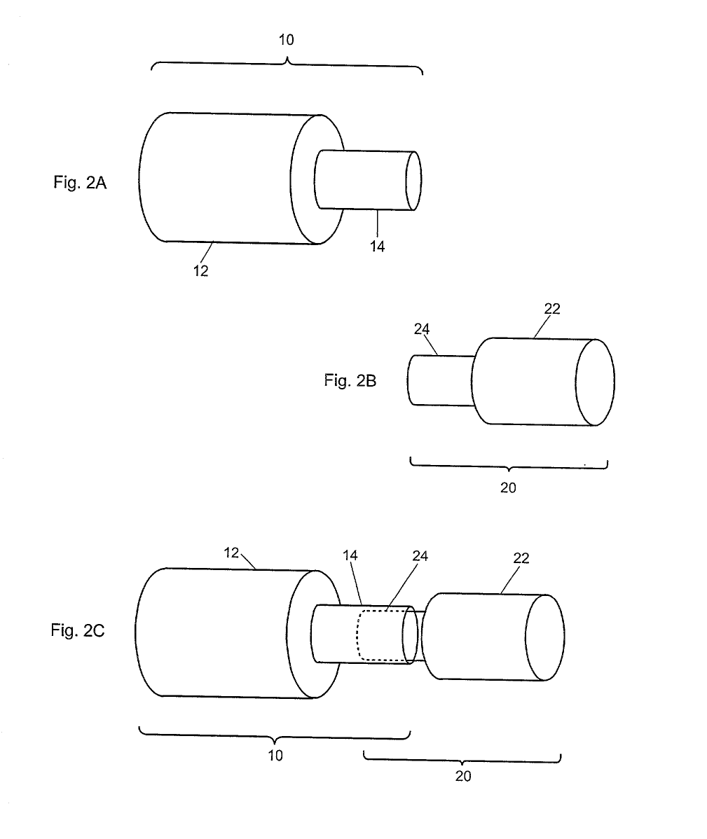

FIGS. 2A-2C are schematic illustrations of an exemplary anchor 10 (FIG. 2A), an exemplary reservoir 20 (FIG. 2B), and an exemplary drug delivery device in which the components are locked together (FIG. 2C). In FIG. 2A, the anchor 10 comprises a first element 12, connected to a second element 14. First element 12 is adapted for radial interference fit with the inner wall of an intact blood vessel. Second element 14 forms a receptacle for mating with a reciprocal locking member of reservoir 20. In FIG. 2B, the exemplary reservoir 20 comprises a first element 24 connected to a second element 22. The first element 24 defines a locking member that engages a reciprocal locking member of the anchor 10. The second element 22 also contains a wall, at least a portion of which defines an inner volume for retaining the drug. In FIG. 2C, the anchor 10 is locked to reservoir 20. The second element of the anchor 14 engages and locks the first element of reservoir 24.

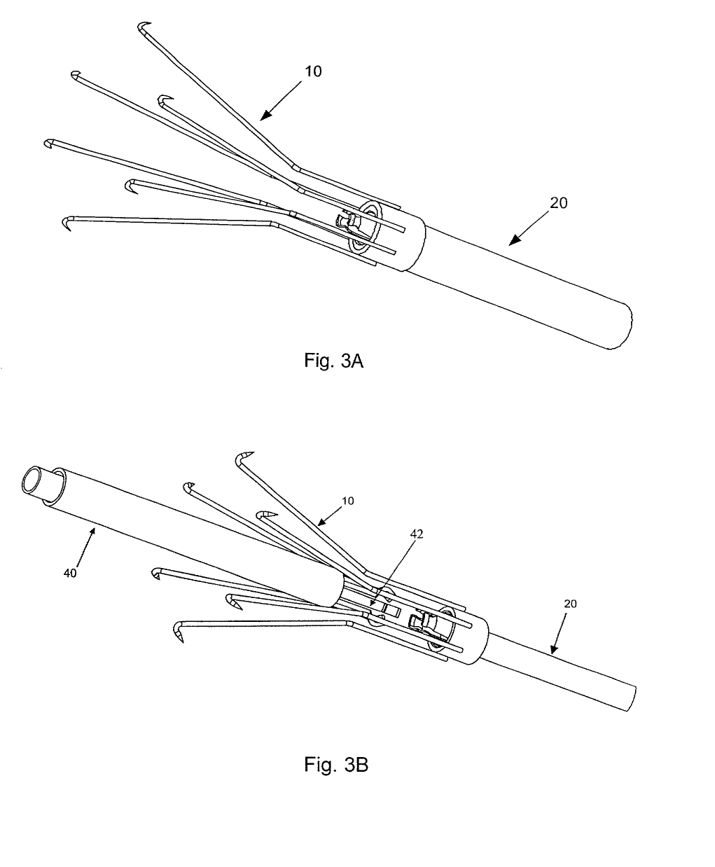

FIG. 3A is a three-dimensional illustration of the device of the invention. In FIG. 3A, anchor 10 is shown engaged to reservoir 20. In FIG. 3B an introduction catheter 40 and a grabbing device 42 disposed within catheter 40 are shown in relation to interlocked anchor 10 and reservoir 20.

Additional designs and design considerations can be found in copending U.S. patent application Ser. No. ______, filed on even date herewith, entitled “Intravascular Blood Conditioning Device and Use Thereof,” and assigned attorney docket number NPH-005, which claims priority to and the benefit of U.S. Ser. No. 60/250,431. The entirety of each of these applications is incorporated herein by reference.

The Anchor

The art is replete with anchors useful in the practice of the invention. Useful anchors are characterized by their ability to be immobilized within the lumen of a blood vessel without occluding or preventing blood flow through the blood vessel, while still providing, as such or after modification, a secure and flexible way to retain the reservoir.

Commercially available embolism anti-migration filters and stents represent exemplary anchors which although lacking locking mechanisms are useful in the practice of the invention. Stents are used routinely by medical practitioners to increase the internal diameter of blood vessels to restore or maintain patency. Blood clot anti-migration or vena cava filters also are used routinely by medical practitioners but are used to prevent the migration of potentially life threatening blood clots within the vasculature. Blood clot anti-migration filters typically are designed to be implanted and anchored within the lumen of a blood vessel. When implanted, the anti-migration filters permit blood in the vessel to pass by while simultaneously trapping blood clots. Commercially available anchors may be used as is or preferably are adapted to further include a locking mechanism that can engage a reciprocal locking member on the reservoir.

The art is replete with helical, cylindrical and/or tubular stent designs capable of modification for use in the instant invention. For example, the stents disclosed in U.S. Pat. Nos. 5,370,691, 5,591,230, 5,651,174, 5,899,935, 5,895,407, 6,107,362, 6,207,516, 6,030,414 and 6,036,725 may be modified to receive and/or engage a drug containing a reservoir. Furthermore, a variety of percutaneous catheter and guidewire systems may be used to introduce and deploy at a desired location stents useful in the practice of the invention (see, for example, U.S. Pat. Nos. 5,891,154 and 6,027,520).

A variety of blood clot anti-migration filters useful in this invention are known in the art and are available commercially. For example, blood clot anti-migration filters described in U.S. Pat. Nos. 4,817,600 and 5,059,205, are available from Medi.Tech®, Boston Scientific Corporation, MA, and are particularly well suited to form the basis for an anchor element required for the practice of the invention. In particular, these filters are designed to provide maximal entrapment area for trapping blood clots while maintaining patency of the blood vessel after trapping emboli. For example, the geometry of the cone-shaped filters permits filling to 80% of its depth before the cross-sectional area is reduced by 64%, and that at least 80% of the depth of the filter can be filled without development of a significant pressure gradient across the filter. The spacing between the six legs of these filters ensures the trapping of emboli greater than 3 mm (Greenfield et al. (1989) “Venous Interruption” Chapter 68, pp. 929-939 in HAIMOVICI'S VASCULAR SURGERY PRINCIPLES AND TECHNIQUES THIRD EDITION, Appleton and Lange, Norwalk, Conn./San Mateos, Calif.). Accordingly, the filters may be used as such to capture a drug-containing reservoir greater than 3 mm in diameter. Other useful blood clot anti-migration filters useful, either as is or after modification by inclusion of an interlocking mechanism are described, for example, in U.S. Pat. Nos. 4,494,531, 4,781,177, 4,494,531, 4,793,348, 4,832,055, 5,152,777, 5,350,398, 5,383,887, 5,720,764, 6,059,825, 6,080,178, and 6,126,673. Also, it is contemplated that other blood clot anti-migration filters, such as those described in Greenfield (1991) in VASCULAR SURGERY, A COMPREHENSIVE REVIEW, Moore, ed. W. B. Saunders Co., Philadelphia, London, Toronto, Montreal, Sydney, Tokyo pp. 669-679, including, for example, Nitinol filters; Gunther filters; Venatech filters; Amplatz filters; and birds nest filters, likewise may be useful in the practice of the invention.

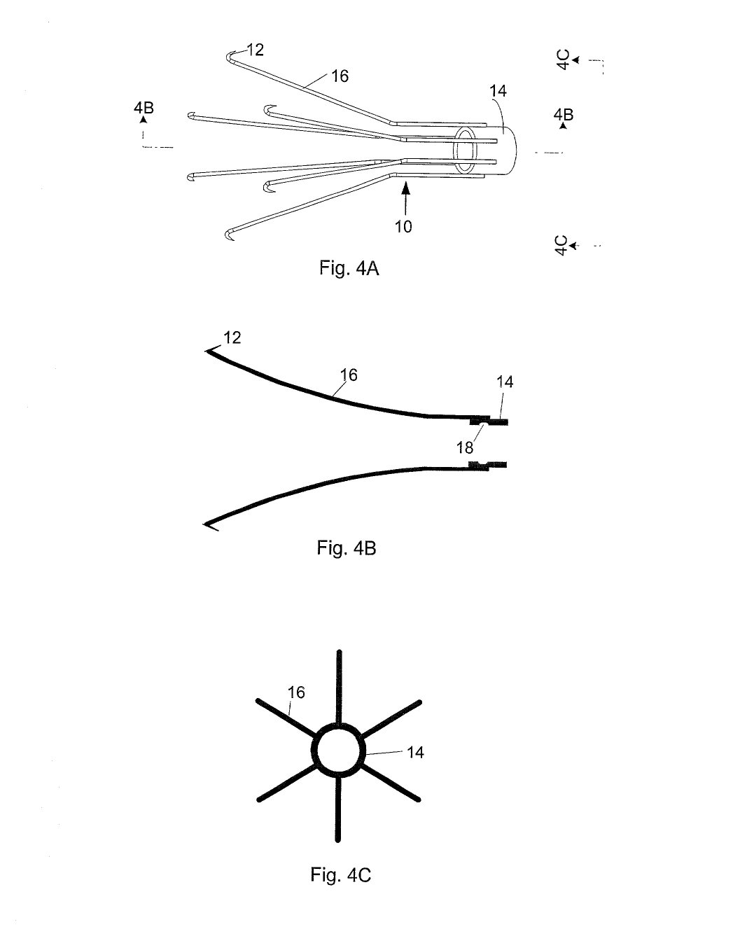

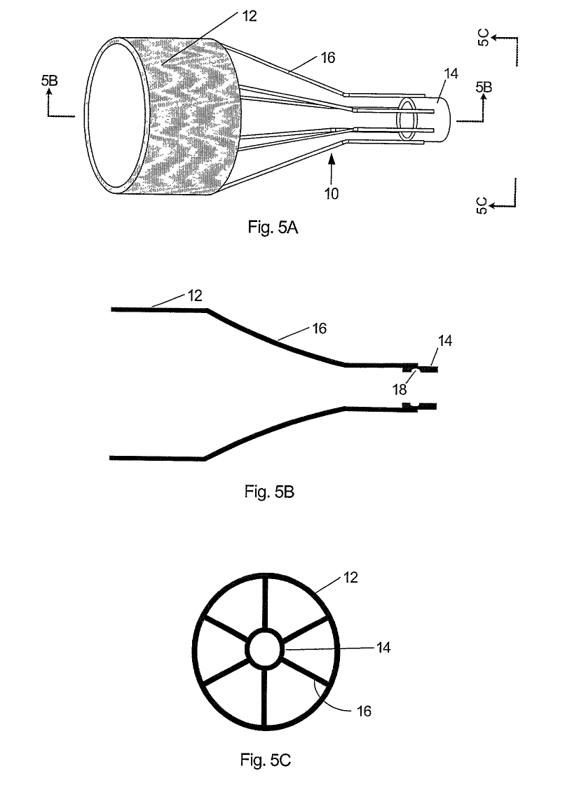

Although commercially available anti-migration filters can be used in the device of the invention, it is preferable that the anchor incorporate a locking mechanism to engage the capsule (see, FIG. 4). Consequently, currently available anti-migration filters typically can be used without further modification. On the other hand, commercially available stents typically do not possess a means for capturing a capsule. However, such stents can be modified, for example, by incorporating an extension comprising legs and a receiving member (see, FIG. 5). Alternatively, unmodified stents can be used as such if, for example, the drug containing reservoir comprises legs with appropriate hooks or barbs that engage a blood contacting surface of the stent. The primary benefit of using such a stent is to spread the force applied by the hooks/barbs to a wide surface area and thus minimize the risk of cartridge migration and to provide the means for repeated implantation/retrieval of the cartridge, while avoiding injury to the vessel wall.

It is preferable, however, that new anchors incorporating locking heads, such as the anchor element shown in FIGS. 4 and 5, are designed and manufactured to better fit the requirements of the present invention. The anchor element may be synthetic or metallic. Preferably, the anchor is made from titanium due to its light weight, strength and biocompatibility.

Two preferred anchors useful in the practice of the invention are presented in FIGS. 4 and 5. In particular, FIG. 4 shows in more detail the anchor element shown in FIG. 3. In FIG. 4A, anchor 10 comprises a head 14 and a plurality of resilient, typically metallic legs 16 extending therefrom. The end of the legs distal to the head comprise hooks or barbs 12 disposed outwardly to engage an inner wall of the target blood vessel. FIG. 4B shows in cross section, head 14 incorporating a locking mechanism 18 which, as described in detail below, is used to engage a reciprocal locking mechanism on the reservoir. FIG. 4C shows in top plan view legs 16 extending radially from head 14. The hooks or barbs 12 of FIG. 4A correspond to first element 12 of FIG. 2A, and head 14 of FIG. 4A corresponds to the second element of FIG. 2A. Leg 16 in FIG. 4A corresponds to a third element that connects the first element (hook or barb) 12 to the second element (head) 14.

An alternative anchor design is shown in FIG. 5. In FIG. 5A, the anchor comprises a head 14 and a plurality of legs 16 extending from head 14 at one end to a stent 12 at the other end. Stent 12 can be a self-expandable stent or can be deployed with the aid of a balloon, or can be any other stent design known in the art. FIG. 5B is a cross-sectional view of the anchor shown in FIG. 5B and shows the spatial relationship of stent 12, legs 16 and head 14, as well as a locking mechanism 18 incorporated in head 14. As described below, the locking mechanism engages a reciprocal locking mechanism of the reservoir. FIG. 5C is a top plan view of the anchor shown in FIG. 5A and shows the spatial relationship between head 14, legs 16 and stent 12.

The primary difference between the anchors shown in FIGS. 4 and 5 is the way in which each anchor is adapted to contact and engage the inner wall of a blood vessel. In the anchor shown in FIG. 4, the outwardly extending barbs may be preferable for implantation inside a vein. This system takes advantage of the relatively low venous blood pressure to minimize the contact area and thus possible negative interaction between vessel and implant. On the other hand, in the anchor shown in FIG. 5, a stent may be preferable for implantation inside an artery, i.e., a high pressure blood vessel. This system takes advantage of the large contact area between the stent and blood vessel ensuring that hydrodynamic forces applied to the implant are spread over a large surface area, thereby minimizing the potential for arterial wall injury or anchor migration.

The Reservoir

The drug delivery reservoir can be any drug containing element that can be immobilized in a blood vessel that, once implanted, releases the drug gradually over time into the systemic circulation. In a preferred embodiment, the reservoir is locked in place to the anchor via a locking mechanism. It is contemplated that any drug of choice may be delivered intravascularly using the device of the invention.

Upon implantation, the reservoir is held securely in place via the immobilized anchor. A reservoir of appropriate design can be introduced into the bloodstream upstream of the anchor which is then transported downstream by blood flow until it is captured passively by the preimplanted anchor, irrespective of the presence or absence of an appropriate locking mechanism between anchor and reservoir. In a preferred embodiment, however, the anchor and reservoir have interconnecting locking mechanisms so that the reservoir can be locked securely in place with the anchor. The incorporation of a locking mechanism can obviate the requirement of introducing the reservoir upstream of the anchor. Thus, use of a locking mechanism enables the implantation of heavier reservoirs for which gravitational forces are significant in comparison to the applied hydrodynamic force. The locking mechanism preferably is designed to permit the capture and engagement of the reservoir and to permit the release of the reservoir.

There are a number of ways to removably attach the reservoir to the anchor, in situ, via mechanical fastener methods, either with or without an interference fit. For example, an outer wall portion of the reservoir can be sized to provide a radial interference fit with a bore or collar in the anchor formed by compliant resilient members, such as cantilevered beams, expandable mesh strands, one or more spring loaded devices or levers, and the like. Alternatively or additionally, the device may comprise a positive mechanical interlock with mating male and female portions, as are known to those skilled in the art of mechanical fastening. Examples include, but are not limited to, threaded members, bayonet retention fittings, ratchet tooth locking latch clamps, and the like. Attachment and/or removal of the reservoir may be accomplished by rotation, translation, or a combination of rotation and translation. Additionally, a catheter can employ an end effector configured to actuate a structure on the reservoir and/or the anchor to facilitate attachment and/or removal, for example, by temporarily expanding a bore, constricting a wall, displacing a latch, opening or closing a clamp, and crimping a compliant member.

The device of the current invention can be used to deliver a variety of drugs into the systemic circulation. It is contemplated that the device of the invention will be particularly useful in the administration of labile drugs, such as drugs sensitive to hydrolysis (for example, prostacyclin), drugs incompatible with stomach acids (for example, protein) or drugs metabolized by tissues before they reach the target site (for example, first pass metabolites). Furthermore, the device of the invention can provide targeted delivery of drugs to the tissue of interest, such as if the device is placed upstream of the target tissue (for example, administration of antiarrhythmic or anticoagulation drugs to the heart, antithrombotic drugs to a prosthesis, antineoplastic drugs for targeted chemotherapy, and antisuppressive drugs to an organ transplant), thereby achieving high local concentrations concurrent with low systemic level. Furthermore, the device of the invention can be used to administer drugs that are toxic if delivery results in high local concentrations (for example, for the delivery of vancomycin, which is detrimental to muscle tissue if administered via intramuscular injection). Furthermore, the device of the invention can be used to deliver drugs useful in treating blood-related disorders, for example, for the administration of factors VIIa, VIII, and IX for hemophilia. Furthermore, the device of the invention can be used to deliver drugs that typically are administered via indwelling catheters, thus offering increased safety from infection. Furthermore, the device of the invention can be used to deliver drugs that preferably are administered frequently (even continuously) and/or in a tightly controlled fashion and/or for a long periods of time (for example insulin or contraceptives). Furthermore, the device of the invention may can be used to deliver drugs to patients who may have difficulty following the recommended delivery schedule, such as young or elderly patients, or for whom drug administration constitutes a degradation of quality of life. Furthermore, the device of the invention can be used to deliver drugs for which other delivery routes are less attractive in view of, for example, equipment requirements, necessity and availability of trained healthcare personnel, required hospitalization, and drug bioavailability and formulation cost.

It is contemplated that the drug delivery device of the invention will be useful in the delivery of natural or synthetic protein therapeutics, such as hormones, activation factors for hormones, enzymes, and antibodies. The device can be used to deliver, for example: Factor VIIa, Factor VIII and Factor IX, protein C and protein S, or anti-thrombin III for the treatment of coagulation disorders, for example, hemophilia or thrombogenic states; hormones such as insulin or somatotropin for hormone replacement therapy (for insulin-dependent diabetes mellitus or growth failure) or reproductive hormones (e.g., for birth control, fertility, or treatment of disorders such as prostate cancer or endometriosis); enzymes to provide lost function due to insufficient de novo synthesis or synthesis of defective enzyme, for example, glucuronosyltransferase or α1-antitrypsin to treat the hepatic diseases Crigler-Najjar or α1-antitrypsin deficiency; enzymes such as phenylalanine hydroxylase to treat metabolic disorders, such as, phenylketonuria; and antibodies, for example, monoclonal antibodies, such as, infliximab and trastuzumab or polyclonal antibodies, such as, antithymocyte globulin, to treat immune disorders and inflammatory disorders.

It is contemplated that the drug delivery device of the invention will be useful in the delivery of agents with vasodilating and cytoprotective properties such as prostaglandins, for example, delivery of PGI₂ (epoprostenol) and its analogs, such as, iloprost (ilomedin) and uniprost (UT-15), in particular for the treatment of primary pulmonary hypertension, but also for the treatment of secondary pulmonary hypertension, perpheral vascular disease, Raynaud's syndrome, systemic sclerosis, and organ trauma (Badesch et al. (2000) ANNALS OF INTERNAL MEDICINE 132:425-434; Higenbottam et al. (1998) HEART 79: 175-179).

It is contemplated that the drug delivery device of the invention will be useful in the delivery of cardiovascular drugs including inotropic drugs, such as dobutamine, milrinone, dopamine, amrinone and enoximone (see, for example, Harjai et al. (1997) CHEST 112:1298-1303; Olivia et al. (1999) AMERICAN HEART JOURNAL 138:247-253; Sindone et al. (1997) AMERICAN HEART JOURNAL 134-889-900; Cesario et al. (1998) AMERICAN HEART JOURNAL 135:121-129); β blockers, such as metoprolol, bisoprolol, carvedilol (Hjalmarson et al. (2000) JAMA 283:1295-1302); diuretics, such as torasemide and furosemide (Liguori et al. (1999) EUR. J. PHARMACOL. 55: 117-124); antiarrhythmic agents, such as, amiodarone (Deedwania et al. (1998) CIRCULATION 98:2574-9); vasodilators, such as, minoxidil and nitroprusside (Masuyama et al. (1990) J. AM. COLL. CARDIOL. 16:1175-85); nitric oxide generators, such as, molsidomine (Lehmann et al. (1995) EUR. J. CLIN. PHARMACOL. 48:109-114); platelet inhibitors, such as, tirofiban, abciximab and eptifibatide (Heeschen et al. (1999) LANCET 354:1757-62); antithrombotic and thrombolytic agents, such as, warfarin, plasminogen activator (PA), such as, alteplase (t-PA) and reteplase (r-PA), and urokinase (Li-Saw-Hee et al. (1998) CIRCULATION 98:2574-9); and anticoagulants, such as, heparin or hirudin (Meyer et al. (1994) CIRCULATION 90:2474-80).

It is contemplated that the drug delivery device of the invention will be useful in the delivery of antibiotics, for example, penicillins (for example, ampicillin, methicillin, nafcillin), cephalosporins (for example, cefepime, ceftazidime, ceftriaxone, cefonicid, and cefazolin), aztreonam, imipenem, vancomycin, clindamycin, macrolides (for example, erythromycin, clarithromycin, azithromycin), aminoglycosides (for example, gentamicin, kanamycin), quinolones (for example, temafloxacin, ofloxacin), metronidazole, amphotericin B, for the treatment of various bacterial and/or fungal infections (see, for example, PRINCIPLES AND PRACTICE OF INFECTIOUS DISEASES, FOURTH EDITION by Mandell, G. L., Bennett, J. E., and Dolin, R. eds. Churchill Livingstone, 1995; OUTPATIENT PARENTERAL ANTIBIOTIC THERAPY MANAGEMENT OF SERIOUS INFECTIONS PART II; AMENABLE INFECTIONS AND MODELS FOR DELIVERY, Proceedings of a Symposium Held on Jan. 26 and 27, 1993, Sonoma, Calif., Hospital Practice, Symposium Supplement, Volume 28, Supplement 2, HP Publishing Company).

It is contemplated that the drug delivery device of the invention will be useful in the treatment of carcinomas via delivery of anti-neoplastic drugs, such as, 5-fluorouracil (5-FU), a pyrimidine antimetabolite that achieves wide-spectrum antineoplastic action by inhibiting thymidylate synthase (TS) and interfering with RNA synthesis and function (Kim et al. (1999) INT. J. ONCOL. 15:921-926; Okuda et al. (1999) ONCOL. REP. 6:587-591); as well as agents used preferentially against specific tumors, for example, streptozocin for treating pancreatic cancer, tamoxifen for treating estrogen-receptor positive tumors, such as, breast cancer, topotecan for treating lung cancer, and sodium iodide (¹³¹I) for treating thyroid cancer.

It is contemplated that the drug delivery device of the invention will also be useful in the delivery of a central nervous system agent, for example, an anticonvulsant, for example, clonazepam or fosphenytoin, an antipyretic or an analgesic, for example, acetaminophen; an anti-migraine medication, for example, imitrex; an immunomodulating compound, for example, an anti-TNF agent like etanercept, or an immunosuppressive drug, for example, mycophenolate, an anti-inflammatory agent, for example, interferon γ or a cytokine, for example, interleukin-10 (IL-10) and interleukin 13 (IL-13); an anti-obesity agent, for example, leptin; an antilipemic agent, for example, a competitive inhibitor of HMG-CoA reductase, for example, atorvastatin; an anti-emetic agent, for example, cisapride and metoclopramide; and a chelating agent, for example, the iron-chelator desferoxamine.

In another embodiment, the device comprises an integral anchor and reservoir. The reservoir can be loaded with drug prior to, or after implantation into a blood vessel. In this type of embodiment, the reservoir comprises an integral anchoring mechanism comprising, for example, one or more barbs, hooks, or stents, for attaching the reservoir to an inner wall of an intact blood vessel. An exemplary design for such a device may be found, for example, in copending U.S. patent application Ser. No. ______, filed on even date herewith, entitled “Intravascular Blood Conditioning Device and Use Thereof,” and assigned attorney docket number NPH-005. It is contemplated that the cartridge described therein may be replaced with the reservoir described herein.

The implanted sustained drugs delivery device of the invention is capable of delivering pre-selected drug over a prolonged period of time, preferably in range of weeks, for example, one, two, three or four weeks, more preferably in the range of months, for example, two, three, four, five, six, seven, eight, nine, ten, eleven, or twelve months, and in some cases in the range of years, for example, one, two, three, four or five years. The drug delivery device of the invention delivers therapeutically effective amounts of the drug into systemic circulation over the desired period of time. Furthermore, it is contemplated that the drug delivery device of the invention may be used to deliver one or more drugs simultaneously into the systemic circulation. The reservoir typically has an inner volume capable of delivering the requisite amount of drug over an appropriate period of time. The inner volume may range from about 10 μL to about 30 mL, more preferably from about 25 μL to about 10 mL, and most preferably from about 50 μL to about 2 mL.

A reservoir useful in the practice of the invention can be an active delivery system in which drug is delivered, for example, via pump action, or a passive delivery system in which drug is delivered, for example, by diffusion and/or convection. Both classes of reservoir are described in more detail below.

1. Active Drug Delivery

Two general classes of reservoirs capable of active drug delivery include chemical pumps and mechanical pumps.

(i) Chemical Pumps

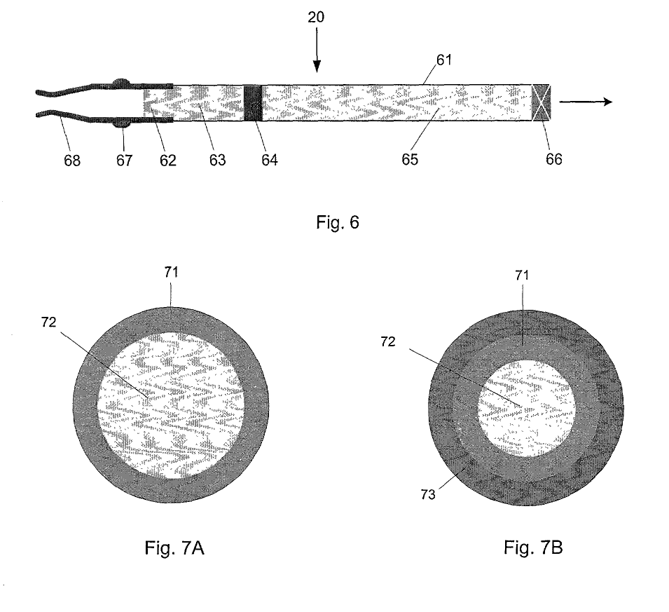

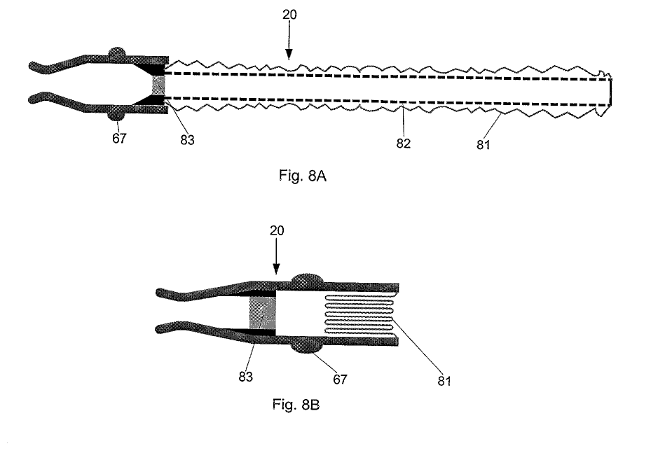

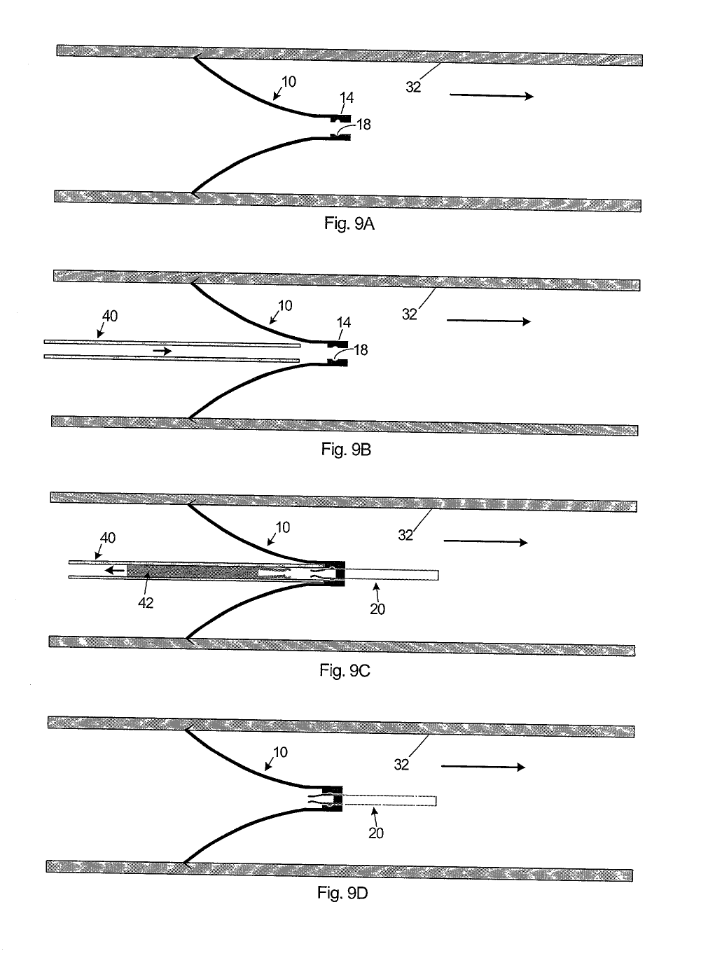

FIG. 6 illustrates a conventional chemical pump. Conventional chemical pumps are available commercially and can include osmotic pumps. It is contemplated that any implantable osmotic pump dimensioned for insertion into a blood vessel of an animal and capable of functioning in that environment can be used in the practice of the invention.

Osmotic delivery systems are available commercially and can be adapted for use with the present invention. Exemplary commercially available osmotic pumps are sold under the tradenames DUROS®, available from Durect Corporation (Cupertino, Calif.), and ALZET®, available commercially from ALZA Scientific Products (Mountain View, Calif.). The DUROS® implant, for example, once implanted in situ, can continuously deliver a pre-selected drug into an animal for up to one year.

FIG. 6 illustrates an exemplary reservoir 20 based on an osmotic pump. The osmotic pump is defined at least in part by a wall 61, for example, a titanium alloy cylinder, that has a first end and a second end. The pump comprises, from the first end to the second end, a semi-permeable membrane 62, an “osmotic engine” 63, a piston 64, pre-selected drug 65, and a delivery orifice 66. When implanted, water permeates the semi-permeable membrane 62 inducing swelling of the “osmotic engine” 63. During operation, the osmotic engine, when it swells, pushes piston 64 in a direction from the first end to the second end which in turn pushes the pre-selected drug 65 through the delivery orifice 66 and out into the blood stream. Because this type of osmotic pump enables the incorporation and delivery of a drug while shielding the drug from the surrounding fluid, it can be used to deliver labile drugs, such as those sensitive to hydrolysis. Furthermore, by choice of an appropriate membrane and/or osmotic engine, it is possible to prolong drug release over periods ranging from one week to more than a year. In particular, currently available DUROS® pumps reportedly can deliver up to 200 mg of pre-selected drug at rates as low as 0.5 μL per day.

As further depicted in FIG. 6, the reservoir 20 optionally can include an interlocking mechanism 67. For example, an interlocking mechanism may be attached to a DUROS® pump that engages a reciprocal interlocking mechanism of the anchor. Furthermore, reservoir 20 may be adapted to include a seizable element 68, that can be seized by a grabber element to facilitate introduction of the reservoir into a recipient and/or removal of the reservoir from the recipient. During operation, by grabbing the exposed end of seizable element 68, the radial dimension of interlocking mechanism 67 can be constricted to facilitate engagement into and/or withdrawal from a reciprocal groove type interlocking mechanism disposed on the anchor.

In another embodiment, the reservoir itself may be adapted to include components of the anchor that permit the reservoir to bind or engage the inner wall of the intact blood vessel. For example, the reservoir may itself comprise a stent or stent-like mechanism or barbs or hooks to engage the inner wall of the blood vessel. This type of reservoir configuration, therefore, obviates the need for a separate anchor.

U.S. Pat. No. 4,685,918 discloses a lipid-based osmotic pump useful in delivering agents with low water solubility. The pump comprises an inner core compartment of active agent, lipid carrier and osmotic agent surrounded by an enclosing wall material. The core having the property that, at body temperature, the lipid becomes or is in a fluid state and retains the active agent in a dissolved or suspended state. The wall consists of one or more polymer layers with the innermost layer being wetted by the lipid in preference to the aqueous solution of the osmotic agent. The wall constitutes a layer that is water permeable. The lipid carrier containing the active agent is released from the system via pores in the wall as a result of a build up of hydrostatic pressure based upon an influx of water into the core.

U.S. Pat. No. 4,777,049 discloses an osmotic delivery system comprising a wall formed of a semi-permeable membrane that is permeable to the passage of an exterior fluid and substantially impermeable to the passage of a therapeutic agent. The membrane defines a compartment that contains the therapeutic agent and a modulating agent. Influx of exterior fluid creates hydrostatic pressure that forces the therapeutic agent through a passageway through the wall and out of the device.

U.S. Pat. No. 5,035,891 discloses a sustained release implant. The implant comprises a semi-permeable membrane that encloses a therapeutic agent, an osmotic agent of solid hydrophilic polymer and an agent that solubilizes the therapeutic agent. The membrane is permeable to the therapeutic agent but not the solubilizing agent and thus offers the advantage of sequestering the solubilizing agent that may potentially be harmful if released into the host. An increase in osmotic pressure caused by influx of fluid causes the therapeutic agent to be expelled from the device.

(ii) Non-Chemical Pumps

Mechanical pumps have been used successfully ex vivo and in vivo. For example, in the case of the implantable artificial heart, a mechanical pump provides the high blood flow rates required to replace the function of the failing native organ. More recently, microaxial blood pumps that fit inside a blood vessel can augment the flow of blood through diseased tissues. For example, studies suggest that a microaxial blood pump can be implanted into the portal vein to augment the liver blood perfusion of patients suffering liver cirrhosis (Marseille et al. (1998) ARTIF. ORGANS 22: 458).

Recent advances in micro-electromechanical systems (MEMS) technology have led to the development of micropumps for use in a variety of applications, including implantation (see, for example, U.S. Pat. No. 5,788,468). Micropumps of sizes less than 2 mm diameter are already available commercially. Such dimensions enable the use of micropumps in implantable intravascular drug delivery devices in the place of the osmotic pump systems described above.

These micropumps are small enough to be packaged into drug delivery cartridges that can be implanted with the aid of standard catheters, such as the 12 French catheter whose internal diameter is about 3.5 mm. At the same time, these micropumps have enough power to drive drug delivery even for the largest size of intravascular drug delivery systems. The micropump may obtain power from an external energy source through wired connections, for example, through the blood vessel and into the anchor, or preferably, wirelessly such as through an inductive coupling or a radiofrequency link (see, for example, U.S. Pat. Nos. 4,102,344; 4,408,608; 4,673,391; and 6,099,495).

Alternatively, the pump may be self-sustained and comprise, for example, a micromotor, an actuated valve and a power supply required to operate them. For example, it may be powered by small energy cells such as silver oxide cells, or through transducer elements (magnetic or piezoelectric) that generate electricity from the hydrodynamic environment surrounding the cartridge (see, for example, U.S. Pat. No. 3,943,936). The micromotor may be rotating at constant speed thereby delivering the drug at a constant rate, mimicking the zero order response characteristic of an osmotic pump. Furthermore, a microchip may be used to control the micromotor thereby yielding a highly flexible drug delivery pump. The microchip can be pre-programmed so that drugs are delivered in accordance with a desirable time delivery profile, for example, by ramping up/tapering down dosage over time or delivering different amounts at different times. Alternatively, the microchip can be programmed to respond to the input provided by implantable microsensors, for example, to deliver insulin in response to glucose levels, or can be controlled externally, for example, through radiofrequencies or IR signals (see, for example, WO 99/55360) according to the specific response of patient to the treatment regime.

Furthermore, it is contemplated that the device may comprise an anchor and, instead of or in addition to the reservoir, a microsensor for detecting the presence and/or concentration of a particular molecule, for example, insulin, in the systemic circulation. Accordingly, such a device comprises a microsensor immobilized within a blood vessel via an anchor. The information derived from the microsensor can then be relayed to an extracorporeal site for analysis by the requisite medical instrumentation and/or personnel or can be used to control an appropriate drug delivery device whether extravascular or intravascular and associated with the anchor.

With reference to FIG. 6, a mechanical micropump-driven drug delivery reservoir may comprise a battery instead of the membrane 62, and a printed circuit and micromotor/gearhead to replace the osmotic engine 63. Miniature motors less than 2 mm in diameter have already been developed and the art is progressing rapidly. Appropriate micromotors are commercially available, for example, through RMB Miniature Bearings, Inc., of Ringwood, N.J., or from MicroMo Electronics, Inc. of Clearwater, Fla.

The motor can be powered with a commercial battery system, such as the high density, high stability silver oxide button cells found in a miniature electronic device. The energy source may be incorporated as an integral component of the reservoir. Even though the reservoir as a whole would need to be replaced when the battery is exhausted, the capacity of silver oxide cells exceeds considerably the energy requirements of typical drug delivery applications. Alternatively, power to the motor can be provided by a large capacity battery external to the blood vessel via microwires connecting to hooks via which the anchor is attached to the lumen of the blood vessel.

In addition, other mechanical micropumps may also be useful in the practice of the invention. For example, the micromotor/piston assembly can be replaced by a piezoelectric micropump whereby a fluid is pumped by the movement of a solid membrane in response to electrical stimulus (see, for example, U.S. Pat. No. 4,938,742). Alternatively, the driving force required to pump the drug out of the reservoir into the bloodstream may be provided by a pressurized fluid. The desired drug release profile can be programmed into a microchip that controls the supply of voltage to actuated microvalves, for example, piezoelectric valves such as those described in U.S. Pat. No. 4,938,742. Furthermore, U.S. Pat. No. 5,368,588 discloses a parenteral fluid medication pump comprising a reservoir filled with fluid medication. Continuous discharge of drug is accomplished by relaxation of forces within a shrink polymer wall surrounding the drug reservoir.

Thus, it is contemplated that any implantable pump suitable for use in the vascular system of an animal may be used, whether it is driven by osmosis, chemical forces, electricity, magnetism, pressure, hydrodynamics or other physical forces.

2. Passive Drug Delivery

The reservoir may also release drug passively into the systemic circulation. In one embodiment the reservoir is a capsule containing the pre-selected drug. The drug may then diffuse out of the capsule and into the blood circulating around the capsule. The transport of drug out of the capsule further may be facilitated by convective currents, for example, ultrafiltration currents, in the interior of the capsule. Convective transport can impart desirable drug delivery kinetics to the capsule. The capsule facilitates the containment of the drug formulation and thus improves the handling and/or loading characteristics of the capsule and prevents the loss of drug particles and the formation of emboli. The capsule may comprise either a single hollow fiber or a plurality of hollow fibers.

(i) Drug Formulation

In order to achieve passive drug delivery, the pre-selected drug can be formulated to facilitate sustained drug delivery over a prolonged period of time. Different formulations include, for example, (i) encapsulating the drug within a polymer membrane from which the drug diffuses over a prolonged period of time, (ii) encapsulating the drug within a liposome which breaks down over time releasing the drug, (iii) distributing the drug evenly through a matrix polymer, whereby drug is released from the matrix as a result of diffusion and/or polymer erosion; and (iv) forming polymer drug conjugates in which the polymer is degraded over time to release the drug (see, for example, Langer (1998) NATURE 392, Supp. 5-10).

In some embodiments, drug is immobilized within a solid or semi-solid (gel-like support). For example, a drug may be encased within a polymeric casing from which the drug slowly leaches out over time. In another embodiment, drug is associated strongly, through chemical or physical forces, with a biodegradable solid support. In such cases, the rate of release depends, for example, on the rate of the degradation of the polymer.

FIG. 7A illustrates an exemplary capsule comprising a semi-permeable membrane 71 defining an inner volume 72 containing the drug either in solution or in suspension. In this embodiment, the release of drug is controlled by the rate of diffusion of the drug through the pores of the membrane 71, which in turn is controlled by the interaction between the membrane, the drug, and the solvent, and by the membrane transport characteristics such as membrane thickness, porosity, pore size, and tortuosity. The membrane may further be bioerodible so that with time the thickness of the membrane decreases and/or its porosity increases, thereby increasing the diffusivity of the drug. Accordingly, a diminishing concentration of drug in the capsule interior can be compensated by the increase in porosity to maintain the rate of drug delivery.

Composite immobilization matrices may also be employed to shift the rate controlling step and thus achieve desired changes in the rate of drug release. FIG. 7B illustrates another exemplary capsule whereby a semi-permeable membrane 71 defines an inner volume 72. The semi-permeable membrane 71, however, is surrounded by an impermeable but degradable layer 73. This system configuration results in the sustained release of drug following a lag phase during which time the impermeable layer 73 is being degraded. There is no drug release until the impermeable layer 73 of the capsule is eroded at which stage the system develops drug release kinetics achieved by the system shown in FIG. 7A. By varying the material and or thickness of the impermeable layer it is possible to control the drug release lagtime.

In other embodiment, the drug may be encased within a semi-permeable microcapsule that also contains an osmotic fluid. In this case, the drug is prevented from escaping from the capsule. In contrast, water can enter the capsule thereby increasing the internal pressure of the capsule to the point where it bursts releasing the capsule's contents, thereby simulating a bolus delivery of drug. The kinetics of drug delivery in this case depends on osmotic pressure, the burst strength of the capsule, the rate of water diffusion through the cartridge and the amount of drug contained therein. It is contemplated that the skilled artisan may achieve a drug delivery profile where bolus drug deliveries occur at different times by varying the size, thickness, and material of the capsule, the osmotic fluid and the drug concentration.

In other embodiment, drug can be associated with a polymer that releases the drug in response to an external stimulus. For example, the polymer can include magnetic microbeads, such that when the polymer is exposed to an oscillating magnetic field of extracorporeal source, the movement of the beads alters the transport characteristics of the polymer thereby releasing the drug as required. Other polymer systems responsive to ultrasound, electric current, pH, temperature, or local concentrations of biomolecules such as glucose are known in the art and can be useful in the practice of the invention (see, for example, U.S. Pat. No. 6,099,864).

In other embodiment, drug may be associated with micro-electromechanical systems (MEMS) that provide more precise control of drug release kinetics. For example, microscopic versions of the drug formulation depicted in FIG. 7B may be disposed upon a microchip, whereby the function of the impermeable but degradable polymer layer 73 may be replaced by a metallic covering layer that is degraded on demand by the application of a microchip-controlled electric current, such as described in U.S. Pat. No. 5,797,898, so that drug becomes available for passive transport by diffusion or convection.

A combination of the foregoing approaches may be used to achieve desirable drug release kinetics.

(ii) Membrane

Membranes useful in producing preferred capsules are fabricated from a semi-permeable material having pores dimensioned to permit the selective transport, by diffusion and/or convection, of pre-selected drug molecule out of the reservoir and into the systemic circulation. The membranes are selected to permit the drug but not the drug formulation particles or microcapsules to be released into the systemic circulation. Optionally, the membrane is designed to prevent the influx of the host's immune cells, for example, macrophages and lymphocytes, which if allowed to enter the interior of the reservoir may be detrimental to the longevity of the pre-selected drug.

The membrane may be produced from a biocompatible polymer which includes, but is not limited to, polyvinylchloride, polyvinylidene fluoride, polyurethane isocyanate, alginate, cellulose acetate, cellulose diacetate, cellulose triacetate, cellulose nitrate, polyarylate, polycarbonate, polysulfone, polystyrene, polyurethane, polyvinyl alcohol, polyacrylonitrile, polyamide, polyimide, polymethylmethacrylate, polyethylene oxide, polytetrafluoroethylene or copolymers thereof. A summary of commercially available hollow fiber membranes, including methods of manufacture and the names of commercial suppliers, is set forth in Radovich (1995) “Dialysis Membranes: Structure and Predictions,” Contrib Nephrol., Basel, Karger, 113: 11-24.

If enough drug can be implanted in a single hollow fiber to produce a desirable level of the pre-selected drug in the blood stream then the capsule of the invention, preferably comprises a single hollow fiber. Alternatively, if the requisite amount of drug cannot be incorporated into a single hollow fiber then the drug may be placed in a plurality of hollow fibers.

Furthermore, it is contemplated that the performance of the capsule may be enhanced by reducing fibrin and/or platelet deposition on, or thrombus formation around the semi-permeable membrane. It is contemplated that excessive fibrin and platelet deposition on, or thrombus formation around the blood contacting surface of the capsule and/or hollow fibers may create additional boundary layer conditions which affect diffusion of the drug into the surrounding blood stream. This problem may be resolved by improving the hemocompatability of the membrane following the methods, described earlier, for improving the biocompatibility of materials coming in contact with blood.

Although for many applications, reservoir size is not limiting, for example administration of prostacyclin for the treatment of primary pulmonary hypertension or delivery of leuprolide to treat prostate cancer, other potential applications require the administration of large amounts of drug. Such applications require either frequent reservoir replacement or an alternative means of implanting larger drug delivery cartridges less frequently. Alternatively, an empty reservoir can be implanted and then filled with drug in situ. While the size of the empty cartridge is small enough so that it can be implanted upon loading with drug the cartridge expands to a much larger size.

FIG. 8 depicts two exemplary empty reservoirs useful in the practice of the invention. FIG. 8A illustrates a reservoir 20 comprising a flexible permeable membrane 81 built around a solid supporting frame 82, for example a perforated tubular frame. The length of the reservoir is fixed whether empty or loaded while its diameter is substantially that of the supporting frame when empty but, like a balloon, its diameter increases to that defined by the surface area and elasticity of the flexible membrane when loaded. The reservoir further comprises a septum 83 which seals the inner volume of the reservoir but yet permits drug to be loaded into the reservoir once located in situ. FIG. 8B illustrates a second exemplary, empty reservoir lacking a solid support frame. In this type of reservoir, membrane 81 of the empty cartridge 20 is folded inside the cavity defined by at least a solid portion of the reservoir and is released from the cavity outwardly due to the positive pressure generated during the in situ loading of the reservoir's interior volume. The membrane material and dimensions must in this case be selected such that upon loading the membrane, like a balloon, assumes the desired elongated rather than spherical shape and maintains the required strength.

Biocompatability of Anchor and Reservoir

The device of the invention is designed to allow the uncompromised passage of blood around it, and to reduce the possibility of thrombogenic or complement responses elicited by the host against the device. Thus, the size of the device depends upon the size of the blood vessel in which it is to be implanted. For example, the size of the reservoir of the drug delivery device preferably is less than 2 cm in diameter if it is to be implanted into a vena cava having a diameter of 4 cm, which leaves about 75% of the cross-sectional surface area of the vessel free to permit blood flow. The reservoir may be adapted to enhance long-term performance, for example, by optimizing blood flow around the reservoir. Such a design, therefore, provides shear levels around the capsule appropriate to prevent the adhesion of platelets onto the blood contacting surface of the reservoir and/or the formation of thrombus and clot, or stenosis.

A variety of reservoirs having different shapes may be useful in the practice of the invention. A preferred reservoir is described in detail in Example 2. The preferred shape is designed to minimize turbulence in the blood passing the implanted intravascular reservoir. The shape of the upstream end of the reservoir appears to be less critical than the shape of the downstream end of the reservoir. In particular, the downstream end of the reservoir preferably is tapered to an apex so as to minimize wake effect. A variety of shapes for the upstream end of the reservoir may be used, however, under certain circumstances it may be advantageous to use a flow directing member to direct the flow of blood around the cartridge. The flow directing member may be conical in shape with the apex of the member located upstream and the base of the member located downstream relative to the reservoir.

In addition, it is also contemplated that the performance of the device may be enhanced by improving the biocompatibility of all of the device materials that come in contact with blood, whether they are parts of the drug delivery reservoir or the anchor. In this regard, a number of approaches have been developed to improve hemocompatability of biomaterials placed within the systematic circulation (see, for example, Ishihara (1993) “Blood compatible polymers”, in BIOMEDICAL APPLICATIONS OF POLYMERIC MATERIALS, Tsuruta T., Hayashi T., Kataoka K., Ishihara K., Kimura Y. (eds.), CRC Press, Boca Raton, Fla.). These efforts include elimination of protein adsorption by increasing material hydrophilicity, diminishing the blood-material interface by increasing hydrophobicity, inhibiting adhesion and activation of platelets by incorporating microphase separation on the surface of the reservoir, incorporating highly mobile hydrophilic moieties and negative charges that simulate the surface properties of blood vessels, or incorporating biologically active molecules on the surface to inhibit the reaction cascade of biological systems such as the coagulation system. The latter is the most extensively developed approach, whereby heparin can be incorporated into a biomaterial to attain local anticoagulation activity on the surface of the biomaterial. For example, Duraflo II heparin membranes (Bentley Labs, Baxter Healthcare Corporation, Irvine, Calif.) comprise a layer of heparin on the coated surface of membrane which is effective for, at least, several days. See, for example, Hsu (1991) PERFUSION 6:209-219; Tong et al. (1992) ASAIO Journal 38:M702-M706. Furthermore, heparin fragments, prepared from the degradation of heparin in nitrous acid, can be covalently linked by end-point attachment of the heparin to a polyethyleneimine polymer coat (Larm et al. (1983) BIOMAT. MED. DEV. ART ORGANS 11(2&3):161-173, Larsson et al. (1987) ANN N.Y. ACAD. SCI. 516:102-115). This process has been shown to provide effective anticoagulant activity on the surface of biomaterial for several months (Larsson et al. (supra)). It is contemplated that heparinization of the blood-contacting surface of the reservoir may minimize fibrin and platelet deposition and/or thrombus formation.

The resulting reservoir subsequently may be implanted either alone or as a bundle of hollow fibers in combination with the blood permeable element into the vasculature of the recipient. Methods for implantation are discussed below.

Implantation of the Device

The device of the invention can be inserted into the vasculature of the host by a non-invasive or minimally invasive surgical procedure. More specifically, it is contemplated that the devices of the invention may be introduced by a variety of catheter-based devices such as those that have been developed for implanting stents and blood clot anti-migration filters into the vasculature.

For example, U.S. Pat. Nos. 3,952,747, 5,147,379, and 5,415,630, and International No. PCT/US92/08366, describe catheter-based devices and methods for implanting blood clot anti-migration filters into the vasculature of a recipient. Typically, the catheter-based filter insertion instruments comprise: a carrier for supporting a blood clot anti-migration filter in a collapsed, compact state; an ejector mechanism, usually located within the carrier for ejecting the filter at the pre-selected site; and an elongated, flexible tube connected to the carrier for advancing the carrier along the blood vessel to the pre-selected location. Once introduced to the preferred location in the blood vessel, the filter is ejected from the carrier. When self opening and implanting filters are used, the filter is simply ejected from the carrier, whereupon the filter anchors itself to the wall of the blood vessel. If, however, a filter to be manually opened and anchored is used, then the insertion instrument may contain additional means for effecting such opening and anchorage steps.

Filters typically are inserted through the internal jugular or femoral vein by percutaneous puncture. During percutaneous insertion, and after a conventional cavogram, either the jugular or the femoral vein is punctured with a needle and a guide wire inserted into the vessel through the needle. Then, a combined sheath/dilator unit is pushed into the vein over the guide wire until the end of the sheath is located beyond the implant site. While holding the sheath in place, the dilator and guidewire are removed, leaving the sheath behind. The sheath acts as an access to permit the insertion of the introducer catheter, which contains a carrier holding the filter. The sheath is flushed with sterile heparinized saline to prevent potential thrombus formation within the sheath which may occur during insertion of the introducer catheter. The introducer catheter is advanced into, but not beyond the end of, the sheath until the tip of the filter carrier capsule is positioned adjacent to the implant site. Then, the sheath is retracted onto the introducer catheter until the carrier capsule is completely exposed. Then, the filter is pushed out of the carrier capsule by a pusher mechanism, whereupon the legs of the filter spring outward and engage the inner wall of the blood vessel thereby anchoring the filter in position. It is contemplated that the anchor can be implanted by the skilled practitioner following a similar procedure. Once the anchor has been ejected and anchored in the blood vessel, the drug delivery cartridge containing the pre-selected drug likewise may be introduced via the same catheter into the blood vessel at a position upstream of the anchor. Use of anchor and drug delivery cartridge elements featuring a complementary locking mechanism would further enable the delivery of the drug delivery cartridge from either side of the anchor. Then, the introducer catheter can be removed from the vessel through the sheath. Once the introducer catheter has been removed, the sheath also is removed, and the puncture site compressed until homeostasis is achieved.

The procedure for implanting stents follows steps analogous to those described above, especially in the case of self-expanding stents. In the case of stents that do not self-expand, the procedure requires additional steps, as balloon-type catheters typically are used to dilate the contracted stent. Balloons are first dilated to expand the catheter and then are deflated to permit withdrawal of the balloon-type catheter. A variety of stent designs and deployment procedures have been developed and are known to those skilled in the art. Exemplary stent designs and corresponding implantation procedures are disclosed, for example, in U.S. Pat. Nos. 4,655,771; 5,071,407; 5,078,720; 6,113,608; 5,792,172; 5,836,965; 6,113,62; 6,123,723; and 6,136,011.

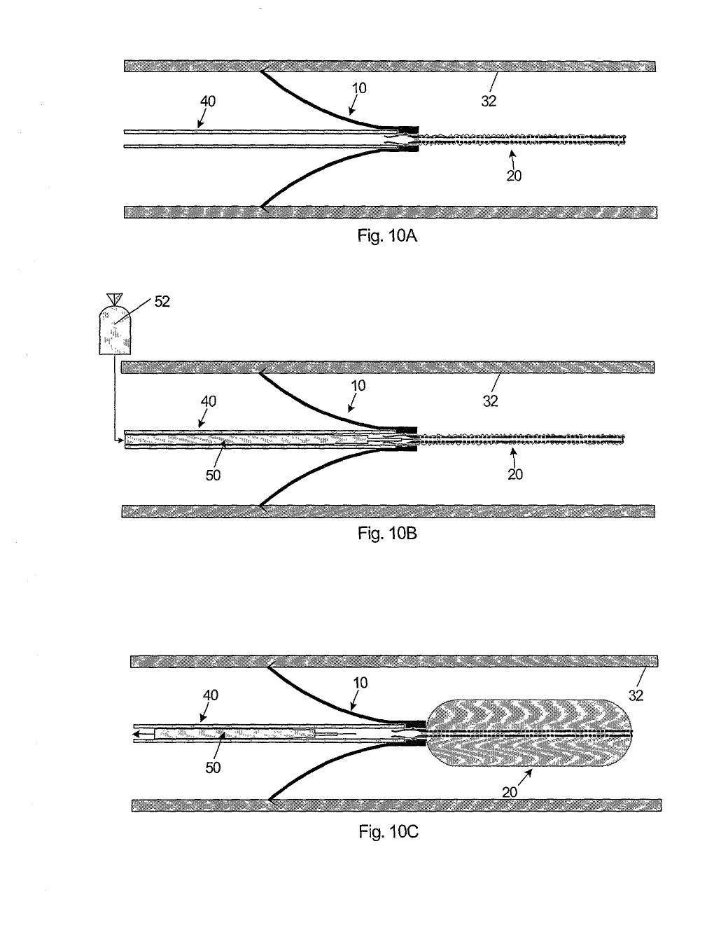

Once immobilized in situ, the reservoir may be introduced into the blood vessel and locked to the immobilized anchor as illustrated in FIG. 9. The direction of blood flow is illustrated by the arrows. FIG. 9A shows anchor 10 immobilized to the inner wall 32 of the blood vessel. The cross-sectional view shows receptacle 14 containing interlocking mechanism 18. FIG. 9B shows the insertion catheter 40 in relation to immobilized anchor 10. FIG. 9C shows reservoir 20 being delivered along catheter 40 via grabbing element 42. Once in place, the grabbing element 42 releases the reservoir 20, and expanding reservoir locking members extend until the interlocking mechanism on reservoir 20 mates with and engages with the interlocking mechanism 18 of the anchor. Once reservoir 20 is engaged, the grabbing element 42 is withdrawn. Thereafter, the insertion catheter 40 is withdrawn leaving the immobilized anchor 10 and reservoir 20 components of the drug delivery device in place (FIG. 9D). This procedure can be reversed to remove the reservoir in the event of complications or upon termination of therapy, or eventually, to replace the reservoir with a new one containing the same or a different drug formulation for continued and/or modified therapy. Furthermore, the foregoing implantation and/or retrieval procedure is flexible and can be used with a wide variety of anchors and/or reservoirs, for example, reservoir based on drug diffusion or convection or active drug delivery, for example, via osmotic and/or electromechanical pumps.

The similar procedure may also be used when the reservoir is empty and is filled with drug when immobilized in situ. FIG. 10 illustrates an exemplary protocol for loading a reservoir with drug in situ. FIG. 10A illustrates anchor 10 immobilized to an inner wall 32 of a blood vessel, and an empty reservoir 20 engaged to the anchor. Insertion catheter 40 is shown in spatial relation to anchor 10 and reservoir 20. FIG. 10B illustrates a conduit 50 disposed within insertion catheter 40. The conduit has at one end a loading device for introducing drug into the reservoir and at the other end it is connected to an extravascular or extracorporeal reservoir 52. The loading device at the end of conduit 50 may comprise a syringe needle that is capable of piercing, for example, a rubber septum disposed in the reservoir through which drug can be introduced into the reservoir. Gravity or an external pump may be used to deliver the drug suspension from extravascular or extracorporeal reservoir 52 into reservoir 20. FIG. 10C shows that once reservoir 20 is filled with drug, conduit 50 can be retracted through catheter 40. After withdrawal of conduit 50 catheter 40 can be retracted leaving the drug delivery device in situ for drug delivery.

Alternatively, the reservoir may be recharged in situ with drug from an extravascular element (for example, a reservoir, a pump, and/or a vascular access port). The extravascular element is connected to, and is in fluid flow communication with, the intravascular reservoir via a conduit. The conduit is connected with the reservoir in association with the anchor at one end and is connected with the extravascular element at the other end. The extravascular element may be located intra or extra corporeally, however, in a preferred embodiment, the extravascular element is located intracorporeally, and, more preferably, subcutaneously. The extravascular element can be refilled periodically, for example, by injection of drug. The drug then flows into and replenishes the intravascular reservoir in association with the anchor. When the extravascular element is a pump, the extravascular, intracorporeal pump can be used to transfer the drug to the intravascular reservoir associated with the anchor and/or store the drug (for example, where the pump has its own reservoir). These embodiments allow for the intravascular reservoir associated with the anchor to be recharged easily, for example, by subcutaneous injection of drug into the extravascular element. The recharging can take place, for example, from about every day to about every four weeks for a period of about one month to about three months.

Also, in another embodiment, no separate intravascular reservoir is in close association with the anchor. However, the extravascular element (for example, a reservoir, a pump either with or without its own reservoir, and/or a vascular access port) is connected and in fluid flow communication with a conduit which enters into the blood vessel where the anchor is located. A portion of the conduit is retained in place by the anchor and drug is discharged directly into the blood stream from an opening in the conduit. The extravascular element is recharged, for example, by subcutaneous injection of drug. This system does not use an intravascular reservoir and relies on the extravascular element to supply drug into the blood vessel. Additionally, surgical access to the end of the conduit is not needed, for example, to suture the conduit in place. Alternatively, instead of using a separate anchor, the conduit may comprise integral engagement means, for example, hooks, barbs, or a stent, for attaching the conduit into the blood vessel. In each of these examples, the anchor or the engagement means immobilize the conduit within the blood vessel and to minimize contact with the wall of the blood vessel. In a preferred embodiment, the outlet of the conduit is immobilized such that the outlet is located approximately in the center of the lumen of the blood vessel.

It is understood that the preferred location for implantation of the device within the systemic circulation, however, may depend upon the intended use of the device. For example, in some situations it is contemplated that it may be desirable to introduce the devices via the femoral or jugular veins and then immobilize the anchor at a location within a natural vein, such as, an inferior vena cava, a superior vena cava, a portal vein or a renal vein. It is understood, however, that based upon clinical circumstances, a physician may determine on a case by case basis the optimal mode for introducing the device as well as the optimal location for anchoring the device. Such judgments are contemplated to be within the scope of expertise of the skilled physician.

Practice of the invention will be still more fully understood from the following examples, which are presented herein for illustration only and should not be construed as limiting the invention in any way.

### EXAMPLE 1

### Implantation Studies

Studies were performed to test the functionality of an intravascular drug delivery device of the invention. These studies were conducted by implanting a device into a dog's vena cava through a venotomy using a catheter delivery system. No negative effects due to the device were observed. The animal's health was not compromised for the duration of the study (21 days). Additionally, implantation did not compromise vena cava patency, or patency of other vessels, for the duration of study. Furthermore, the device itself remained intact and remained at the implantation site (no creeping or migration). Drug release from the device also was verified in vivo using a fluorescently labeled compound.

The devices were constructed by combining drug delivery cartridges (i.e., reservoirs) with anchors. The devices were similar to those described in FIGS. 3A and 3B. In addition, the devices further comprised a flow director between the cartridge reservoir and the anchor. Because this experiment focused on the interaction between the intravascular implant and the host animal, the cartridge reservoir was fixed permanently to the anchor rather than via a coupling system. For the same reason, the device was implanted into the animal via a venotomy rather than using a percutaneous delivery system.

The devices were constructed using an ALZET® osmotic minipump, available commercially from ALZA Scientific Products (Mountain View, Calif.), as the model drug delivery cartridge reservoir. The ALZET® model number 1002, a micro-osmotic pump capable of delivering 0.25 μL/h for 2 weeks, was used in this study. The cartridge reservoir was fixed to the anchor assembly with a rapid cure ethyl cyanoacrylate adhesive (Insta-Cure 3SI-1, available from BSI, Atascadero, Calif.). The coupling of the cartridge reservoir to the anchor was streamlined with a flow director machined out of 0.25 inch diameter PTFE rods. The flow director slid over the head of the anchor and maintained its location through a friction fit. Additionally, the flow director had a generally conical shape with the narrow portion constructed to be located upstream when the device was implanted in situ and the wide portion constructed to be located downstream when the device was implanted in situ. The conical shape allowed the flow director to direct blood flow around the cartridge reservoir. The flow director also was machined at the wide or base end to provide a concave surface complementary to a convex surface of the cartridge reservoir to provide a receptacle for the cartridge reservoir and allow for a good fit and seal between the components. The anchor was either a commercial blood clot antimigration filter (a Greenfield® filter) or a similar straight-limb filter constructed with medical grade 0.015 inch stainless steel (316L) wire. For example, one device was constructed with a 12-F Greenfield® filter as the anchor and a mico-osmotic pump as the cartridge reservoir. These two components were interfaced with a teflon flow director.

During construction, the anchor and flow director were sterilized with ethylene oxide prior to affixing the cartridge reservoir. The cartridge reservoir was purchased sterile. The cartridge was filled with a sterile solution or suspension of the agent to be delivered and assembled aseptically under a laminar flow hood. The filled cartridge reservoir then was affixed to the anchor with the sterile instant cure adhesive, and the complete device assembly placed into a delivery catheter, a sterile PTFE tube with a 5/16 inch inner diameter and a 1/32 inch wall thickness. The size of the catheter was selected so that it would fit easily into the vena cava of the test animals (dogs) while still accommodating the device, allowing the device to glide through it when pushed by a plunger.

Large dogs, weighing approximately 30 kg, were used for the implantation procedure. Prior to surgery, the animals were fasted overnight with water provided ab libitum. Before surgery, the dogs were given an injection of 0.2 mg/kg Butaphenol, 0.05 mg/kg Acepromazine, and 0.01 mg/kg Glycopyrollate as proanesthesia. The animals then were anesthetized via intravenous administration of 200 mg pentothal, intubated, and maintained under anesthesia with 2% isofluorane (balance oxygen).

After the vena cava was exposed, the renal arteries and veins were isolated and occluded. Immediately, the vena cava was cross-clamped to prevent flow and a partial venotomy was performed. The delivery catheter containing the device was inserted into the vena cava through the opening. The device was placed such that the cartridge reservoir was facing downstream. Subsequently, the device was pushed inside the catheter with the aid of a plunger. Following its exit from the catheter, the anchor expanded umbrella-like, engaging the vessel wall. Then, the plunger and catheter were withdrawn, leaving the device implanted in situ. The vena cava section then was closed with 5.0 proline sutures. The clamps and ties were removed and, after careful inspection for bleeding, the abdominal cavity was closed using a three-layer closure with 2-0 Vicryl suture. Post-operatively, animals were given 0.02 mg Bupemex for pain relief as well as 800 mg of Bacterim, an antibiotic, twice daily to prevent infection. After recovery, the animals were returned to their cages. The life of the ALZET® pump used in this study (21 days) provided the upper limit for the implantation period.

Following implantation, vena cava patency was verified by fluoroscopies at fixed time intervals. At the end of the experiment, the animal was euthanized, its abdominal cavity opened, and the revealed internal structures were inspected carefully. The vena cava was removed along with the implanted device, rinsed, and sectioned longitudinally to reveal the implant for evaluation of the host-implant interaction. To evaluate the extent of thrombus formation as a result of the presence of the device in the intravascular space, the heart and lungs were removed and sectioned to determine if thrombi had lodged into blood vessels and occluded them. Heart and lung samples were collected along with samples of cava, liver, and kidney tissue for subsequent analysis for the presence of agents infused through the implanted drug delivery cartridge reservoir.

Blood flow through the vena cava was not compromised by the intravascular implant. Fluoroscopic images taken at 18 days post implantation, the last fluoroscopy performed prior to study termination at 21 days, revealed that blood flow was uncompromised. Flowing blood registered around the drug delivery cartridge reservoir, which appeared symmetrically in the center of the vessel. This unoccluded flow was seen despite the fact that the diameter of the cava (approximately 10 mm) was only slightly larger that the diameter of the implant (approximately 6 mm). A human vena cava is larger, typically larger than about 20 mm in diameter, so patency in humans should be less of a concern. In addition, this fluoroscopic analysis indicated that blood flow around the device was not compromised seriously even in the interior of the anchor and that the device retained its integrity.

After the animal was sacrificed at 21 days, the following observations were made. There was no compromise of the cava wall, no inflammation, and no migration of the device. Also, a portion of the anchor limbs were incorporated into the vessel endothelium, but the cava lumen was clean and free of any adhesions. There was some clotting at the device itself, primarily around areas of stagnant flow (for example between the anchor limbs), but, based on the autopsy, clotting was limited to that area. Finally, there were no signs of clotting or thrombi in any of the analyzed tissues, including the vena cava, heart, and lungs.

Additionally, the strength of engagement between anchor and cava wall was analyzed. During harvesting and longitudinal sectioning of the vena cava to observe the device and cava, all 6 limbs of the anchor were kept engaged to the cava wall. Accordingly, a spring-based force meter was used to pull the anchor apart from the cava wall. The force measured prior to separation exceeded 2 lb_{f} or 10 N. It is contemplated that a measured engagement force would be larger if the vena cava was unsectioned.

The infusion of agents from the cartridge reservoir during implantation also was verified. The ALZET® micro-osmotic pump was loaded with a suspension of 20 nm polystyrene microspheres (Molecular Probes F-87-87). These particles were selected as an indicator because (i) they fluoresce strongly and are thus easy to detect, (ii) they are stable (i.e., they are not degraded or metabolized) and inert, and (iii) they are size-excluded from kidney clearance. At the end of the study, the fluorescent microspheres were observed lodged in all collected tissue sections.

These experiments show that it is possible to introduce a drug delivery device into the vaosculative of a host, and, when introduced, such devices are tolerated by the host. Furthermore, once introduced, the devices deliver the compound of interest into the blood stream of the host.

### EXAMPLE 2

### Flow Studies

The shape of each component of the implantable device preferably is optimized to minimize the degree of interaction between the device and the blood. If stagnant flows and vortices can be reduced or eliminated in the intravascular space in the vicinity of the device, then individual components of blood, for example, circulating platelets, may be prevented from collecting around the device. Furthermore, the residence time of such blood components in contact with the device may be shortened thereby substantially decreasing the potential for clotting. By way of illustration, at a typical flow rate of 2 L/min in an inferior vena cava having a diameter of 2.5 cm, the mean linear velocity of blood is estimated to be 21.3 cm/sec. Accordingly, it is estimated that it would take half a second for blood to flow over a 10 cm long implant. However, the introduction of an implant of substantial size into the vascular space may disturb blood flow considerably and generate areas with eddies and flow stagnation (such areas have been recognized as prone to clotting). It is possible to minimize flow disturbances by streamlining the shape of the implant to yield shapes commonly considered as “aerodynamic.”

The effect of various implant shapes can be visualized using a model flow system that simulates the fluid dynamics of a vena cava containing an implant anchored onto the vessel lumen. In such a model, transparent Tygon tubing can be used to simulate a human vena cava. After a test implant is positioned inside the Tygon tubing, water at room temperature is pumped through the tubing via a peristaltic pump. The flow rate can be controlled so as to achieve fluid dynamic similarity between the model system and a human vena cava (i.e., the Reynolds number in the model system is similar to that calculated for blood flowing inside a human vena cava). Fluid flow can be visualized by introducing a colored dye into the tubing, upstream from the implant model. Dye streamlines reveal the nature of the fluid flow for a particular implant model, which can be recorded with a tripod-mounted motion camera.

By implanting test devices comprising a model cartridge of a poly propylene ¼ inch rod machined to a shape of interest affixed to a model anchor (for example, a 12F Greenfield® filter) into such a model system, it was found that rounding of the edges of the model cartridge was usefull to minimize eddies and areas of stagnant flow. Based on this type of study, the degree of rounding required at the front end of the model cartridge was not as important as that required at the tail end of the model cartridge. A conical shaped flow director with a radial profile and radius similar to the radius of the polypropylene rod was sufficient to provide a preferred shape at the front end. A sharper-shaped tail was helpful in minimizing the formation of a turbulent wake at the rear of the model cartridges. The development of wake was found to be dependent on the relative diameter of the model cartridge and the model vena cava. Where the implant cartridge was less than a third of the diameter of the tubing, it was found that a sloping tail design with the tail extending for a distance approximately equal to two diameters of the model cartridge's main body could be sufficient to eliminate wake formation. In contrast, if the tail end of the model cartridge was not shaped (for example, the model cartridge had a pure cylindrical shape), a wake with two symmetrical eddies could be formed. Based on studies of this type, the cartridge shape preferably includes a rounded or sloping tail design extending to an apex, where the distance from the body of the cartridge to the apex of the tail is equivalent to approximately one to approximately three diameter lengths of the body of the cartridge.

### EXAMPLE 3

### Delivery of Prostacyclin Analogs for Treating Primary Pulmonary Hypertension

Primary pulmonary hypertension is an extremely serious, currently incurable disease associated with high morbidity and mortality rates. The disease is the result of inadequate production of prostacyclin (also known as epoprostenol and prostaglandin I₂ or PGI₂), a molecule that is secreted by endothelial cells throughout the vasculature and plays a major role in the maintenance of blood vessels. Among other effects, prostacyclin is a strong vasodilator and a potent inhibitor of platelet activation and thrombus formation. Insufficient amounts of prostacyclin in the pulmonary blood vessels can lead to their narrowing, resulting in high blood pressure in the pulmonary artery and the inadequate flow and oxygenation of blood in the lungs. Thus, despite having otherwise healthy heart and lungs, patients afflicted with primary pulmonary hypertension cannot function normally. If left untreated, the disease can lead to secondary heart failure. In certain cases, treatment may require lung and heart transplantation. However, in recent years successful treatments based on the administration of prostacyclin and its analogs have been developed. Prostacyclin therapy initially was developed to sustain patients long enough to permit a heart-lung transplantion. Recent reports, however, present encouraging results for patients who have been treated with long-term continuous intravascular administration, with the aid of a portable extracorporeal infusion pump (Shapiro et al. (1997) J. AM. COLL. CARDIOL., 30:343-9) or the stable synthetic analog, iloprost (Higenbottam (1998) HEART 79: 175-179).

The device of the current invention can be used to further improve the therapy of primary pulmonary hypertension by replacing the portable infusion pump/catheter system and prostacyclin or prostacyclin analog reservoir with a completely self-contained device capable of infusing the drug close to the targeted tissue over prolonged periods of time, for example, at least three months. Accordingly, an anchor such as that shown in FIG. 4 may be implanted with the aid of a catheter to the vena cava of a patient. Iloprost, the stable analog of prostacyclin can be loaded into a reservoir, for example, a commercially available DUROS®-type pump. Iloprost, also known under the trade names Endoprost, Ilomedin and Ilomedine, is available from Schering AG (Berlin, Germany) and may be preferable to epoprostenol (also known under the tradename Flolan and available from Glaxo-Wellcome) because of its increased vasodilating action requiring only half dose, its stability and its increased chemical stability (see, for example, Skuballa et al, “Chemistry of stable pro'stacyclin analogs: synthesis of iloprost”, in PROSTACYCLIN AND ITS STABLE ANALOG ILOPROST by Gryglewski and Stock (eds), Springer Verlag, Berlin 1987 and Racz et al. PHARMAZIE (1986) 41:769-771).

Clinical experience with Iloprost treatment of this disorder (Higenbottam (1998) supra) indicates that doses in the range of 0.7 to 3.9 ng/kg/min are required to provide significant therapeutic benefits, with the mean level being 2.1 ng/kg/min, although larger dosages may be required or preferred if they are tolerated by the patients. At average dosage level reported in the aforementioned study, it is estimated that a patient weighing 60 kg would require only 5.4 mg/month. Accordingly, it is contemplated that the DUROS®-type pump can accommodate enough drug solution to treat the patient for several months. Once depleted of Iloprost, a catheter may be inserted as described earlier to retrieve the empty pump and, if required, replace it with a new one. Alternatively, the reservoir may be recharged with drug in situ using a catheter connected at one end to the pump and at the other to an extravascular element (for example, a reservoir, a pump, and/or a vascular access port) capable of containing drug.

It is contemplated that such a device would be capable of delivery Iloprost to a patient suffering from primary pulmonary hypertension in an amount and over a time sufficient to ameliorate the symptoms of the disorder.

Incorporation by Reference

The disclosures of each of the patent documents and scientific articles identified herein are expressly incorporated herein by reference.

Other Embodiments

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

Other embodiments of the invention are within the following claims.

What is claimed is:

1. An intravascular drug delivery device for delivering a pre-selected drug into systemic circulation of an animal, the device comprising:
(a) an anchor immobilizable to an inner wall of an intact blood vessel which, when immobilized in the blood vessel, permits blood in the vessel to pass therethrough; and
(b) a cell-free reservoir containing pre-selected drug, which when introduced into the blood vessel is retained by the anchor and releases the pre-selected drug into blood passing the reservoir.

2. The device of claim 1, wherein the anchor comprises at least one element biased in a radially outward direction when immobilized in the blood vessel.

3. The device of claim 1, wherein the anchor is a stent.

4. The device of claim 1, wherein the anchor comprises an outwardly extending barb.

5. The device of claim 1, wherein the anchor comprises a head and a plurality of barbed filaments attached by one end to the head.

6. The device of claim 5, wherein the anchor is an embolism anti-migration filter.

7. The device of claim 1, wherein the anchor comprises a receptacle for receiving the reservoir.

8. The device of claim 7, wherein the receptacle further comprises an interlocking mechanism for locking the reservoir to the anchor.

9. The device of claim 8, wherein the reservoir further comprises an interlocking mechanism that engages the interlocking mechanism of the anchor for locking the reservoir to the anchor.

10. The device of claim 1, wherein the reservoir comprises a wall at least partially defining an inner volume for retaining the pre-selected drug.

11. The device of claim 1, wherein the reservoir is a pump.

12. The device of claim 11, wherein the pump is an osmotic pump.

13. The device of claim 1, wherein the reservoir is a drug permeable capsule.

14. The device of claim 13, wherein the capsule has disposed therein particles containing the pre-selected drug for release therefrom.

15. The device of claim 10, wherein the wall is a semi-permeable membrane.

16. The device of claim 15, wherein the semi-permeable membrane defines pores of a size sufficient to permit diffusion of the pre-selected drug therethrough.

17. The device of claim 16, wherein the semi-permeable membrane comprises a material selected from the group consisting of polyvinylchloride, polyvinylidene fluoride, polyurethane isocyanate, alginate, cellulose, cellulose acetate, cellulose diacetate, cellulose triacetate, cellulose nitrate, polyacrylate, polycarbonate, polysulfone, polystyrene, polyurethane, polyvinyl alcohol, polyacrylonitrile, polyamide, polyimide, polymethylmethacrylate, polyethylene oxide, polytetrafluorethylene, and mixtures thereof.

18. The device of claim 1, wherein the pre-selected drug is a fatty acid, a cardiovascular drug or a coagulation factor.

19. The device of claim 1, wherein the reservoir comprises a plurality of pre-selected drugs which are released into blood passing the reservoir.

20. The device of claim 1, wherein the reservoir releases the pre-selected drug over a pre-selected period of time.

21. A method of introducing into a blood vessel a drug delivery device for delivering a pre-selected drug directly into systemic circulation of an animal, the method comprising the steps of:
(a) immobilizing an anchor an inner wall of an intact blood vessel, which when immobilized permits blood in the vessel to pass therethrough;
(b) introducing into the blood vessel a cell-free reservoir containing pre-selected drug, such that when introduced into the blood vessel the reservoir releases the pre-selected drug into blood passing the reservoir; and
(c) permitting the reservoir to be retained in the blood vessel by the anchor.

22. The method of claim 21, comprising the additional step of, prior to step (a), introducing the anchor into the blood vessel via a catheter.

23. The method of claim 21 or 22, wherein the reservoir is introduced into the blood vessel by a catheter.

24. The method of claim 21, comprising the additional step of locking the reservoir to the anchor.

25. The method of claim 24, wherein the reservoir is locked to the anchor after the anchor is immobilized in the blood vessel.

26. An anchor for implantation into an intact blood vessel of an animal, the anchor comprising:
a first element adapted for immobilization to an inner wall of the blood vessel, wherein the first element comprises at least one member biased in a radially outward direction when immobilized in the blood vessel; and attached thereto
a second element forming a receptacle for receiving a drug delivery reservoir member of a predetermined configuration.

27. The anchor of claim 26, wherein the first element is located proximal to the second element.

28. The anchor of claim 26, wherein the first element is a stent.

29. The anchor of claim 26, wherein the first element comprises at least one outwardly extending barb.30. The anchor of claim 26, further comprising a third element interposed between the first and second elements for connecting the first and second elements.

31. The anchor of claim 30, wherein the third element comprises a filament.

32. The anchor of claim 26, wherein the second element further comprises an interlocking mechanism for engaging an interlocking mechanism on the reservoir to lock the reservoir to the anchor.

33. The anchor of claim 32, wherein the interlocking mechanism comprises an annular member having an inner wall that defines a bore running therethrough, wherein the inner wall further defines a groove perpendicular to the bore for engaging the interlocking mechanism on the reservoir.

34. A drug delivery reservoir for implantation into an intact blood vessel of an animal, the reservoir comprising:
a first element forming an interlocking mechanism for engaging a receptacle of an anchor immobilizable to an inner wall of an intact blood vessel; and attached thereto
a second element having a wall at least partially defining an inner volume for retaining the drug and defining at least one pore dimensioned to permit the drug retained therein to pass therethrough.

35. The reservoir of claim 34, wherein the first element comprises an annular member having an outer wall, wherein a first portion of the outer wall has a first radial dimension, and a second portion of the outer wall has a second, different radial dimension, wherein the second radial dimension is greater than the first radial dimension.

36. The reservoir of claim 34, wherein the second element is a pump.

37. The reservoir of claim 34, wherein the pump is an osmotic pump.

38. The reservoir of claim 34, wherein the second element is a drug permeable capsule.

39. The reservoir of claim 38, wherein the capsule has disposed therein particles containing the pre-selected drug for release therefrom.

40. The reservoir of claim 34, wherein the wall is a semi-permeable membrane.

41. The reservoir of claim 40, wherein the semi-permeable membrane defines pores of a size sufficient to permit diffusion of the pre-selected drug therethough.

42. The reservoir of claim 34, wherein the drug is a fatty acid, a cardiovascular drug, or a coagulation factor.

43. The reservoir of claim 34, further comprising a plurality of pre-selected drugs for release therefrom.

44. An implantable, intravascular drug delivery device, the device comprising:
(a) an anchor comprising a first element adapted for immobilization to an inner wall of a blood vessel, wherein the first element comprises at least one member biased in a radially outward direction when immobilized in the blood vessel and, in connection therewith, a second element comprising a first interlocking mechanism; and
(b) a reservoir comprising a first element comprising a second interlocking mechanism and in connection therewith a second element having a wall at least partially defining an inner volume for retaining the drug and defining at least one pore dimensioned to permit the drug retained therein to pass therethrough,
wherein the first interlocking mechanism is capable of engaging the second interlocking mechanism to the lock the reservoir to the anchor.
